(19) United States
(12) Patent Application Publication
(10) Pub. No.: US 2012/0232169 A1
(43) Pub. Date: Sep. 13, 2012

(54) HIGHLY MONODISPERSE BRANCHED PEG-LIPID CONJUGATES

(75) Inventors: Nian Wu, North Brunswick, NJ (US); Brian Charles Keller, Antioch, CA (US)

(73) Assignee: BIOZONE PHARMACEUTICALS, INC., Pittsburg, CA (US)

(21) Appl. No.: 13/412,676

(22) Filed: Mar. 6, 2012

Related U.S. Application Data
(60) Provisional application No. 61/449,793, filed on Mar. 7, 2011.

(51) Int. Cl. A61K 47/14; C07C 59/58

(52) U.S. Cl. 514/786; 554/219

(57) ABSTRACT

(PEG)-lipid conjugates and methods of preparation are disclosed herein. Methods of preparation may involve stepwise addition of small PEG oligomers to a glycerol backbone until a desired chain size is attained. Polymers resulting from the syntheses may be highly monodisperse. The resulting polymers may comprise branched polyethyleneglycol (PEG)-lipid conjugates. The present disclosure may provide several advantages such as simplified synthesis, high product yield and low cost for starting materials. The present synthesis method may be suitable for preparing a wide range of conjugates such as PEG lipid conjugates having a glycerol-like central backbone covalently attached to two or more monodisperse PEG chains and a lipid comprising a range of diesters made from fatty acids or bile acids.

This application claims priority to the provisional patent application Ser. No. 61/449,793, entitled “Pure Branched Peg-Lipid Conjugates” filed in the U.S. Patent and Trademark Office on Mar. 7, 2011, by Nian Wu and Brian Charles Keller.

### FIELD OF THE INVENTION

The present invention relates to polyethyleneglycol (PEG)-lipid conjugates. More particularly, the invention relates to synthesis and composition of branched PEG-lipid conjugates with substantially monodispersed PEG chains.

### BACKGROUND OF THE INVENTION

When used as a delivery vehicle, PEG-lipid conjugates have the capacity to improve the pharmacology profile and solubility of lipophilic drugs. They also provide other potential advantages such as minimizing side effects and toxicities associated with therapeutic treatments.

Narrow molecular weight distribution of polymers used for drug delivery is crucially important for biomedical applications, especially if used for intravenous injections. For instance, PEG-8 Caprylic/Capric Glycerides is a mixture of monoesters, diesters, and triesters of glycerol and monoesters and diesters of polyethylene glycol which has a mean relative molecular weight between 200 and 400. The application of PEG-8 CCG for solubilizing many water-insoluble drugs is restricted, and has a dose limit of approximately 6%, for human oral drug formulations due to allergic reactions observed in animals models.

When PEG chains may be produced by free radical polymerization, molecular weight distributions may not be narrowly controlled, especially for chains having molecular weights between about 200 and 2,000 daltons and above. Far less than 50% of the polymers in a batch may have exactly the targeted molecular weight. A narrower distribution may be achieved by total synthesis or size exclusion chromatography, which may result in more of the PEG polymers having a targeted molecular weight, especially for long PEG chains.

Mono-distribution of single chain to 24 PEG subunits or branched chains with up about 72 PEG subunits are commercially available, additional synthetic steps may be used to incorporate them into pharmaceutical and/or cosmetic formulations.

### BRIEF SUMMARY OF THE INVENTION

Syntheses of branched polyethylene glycol (PEG)-lipid conjugates and compositions thereof are presently disclosed. Such syntheses may involve addition of branched PEG oligomers to a glycerol backbone until the desired PEG-lipid conjugate is attained. Polymers resulting from the syntheses may be highly monodisperse. The present synthesis method may be suitable for preparing a wide range of conjugates.

In at least one aspect of the present disclosure, the invention comprises PEG lipid conjugates having a glycerol backbone covalently attached to one or two monodisperse branched PEG chains and one or two lipids. These conjugates may be especially useful for pharmaceutical formulations.

### BRIEF DESCRIPTION OF THE FIGURES

FIG. 1 shows Chemical Structure 1 which illustrates the difference in steric conformation of two such isomers.

### DETAILED DESCRIPTION OF THE INVENTION

Embodiments of the present invention are described herein in the context of synthesis methods, intermediates, resulting compounds, and compounds related to making PEG-lipid conjugates with narrowly defined molecular weights. Those of ordinary skill in the art will realize that the following detailed description of the present invention is illustrative only and is not intended to be in any way limiting. Other embodiments of the present invention will readily suggest themselves to such skilled persons having the benefit of this disclosure. Reference will be made in detail to implementations of the present invention.

In the interest of clarity, not all of the routine features of the implementations described herein are shown and described. It will, of course, be appreciated that in the development of any such actual implementation, numerous implementation-specific decisions must be made in order to achieve the developer's specific goals, such as compliance with application- and business-related constraints, and that these specific goals will vary from one implementation to another and from one developer to another. Moreover, it will be appreciated that such a development effort might be complex and time-consuming, but would nevertheless be a routine undertaking of engineering for those of ordinary skill in the art having the benefit of this disclosure.

When employing PEG-lipid conjugates as drug delivery vehicles, it is becoming increasingly important to use well-characterized and highly pure conjugates. For example, U.S. Pat. No. 6,610,322, which is incorporated herein by reference, teaches that varying the length of PEG and acyl chains affects the packing parameters of the conjugates which in turn determine whether compositions of PEG-lipid conjugates form liposomes or not. In addition to affecting the physical structure of drug formulations, the choice of lipids and PEG sizes may have significant effects on pharmacokinetics and stability when formulating specific drug compounds with PEG-lipid conjugates. Therefore, uniform batches of conjugates having monodisperse PEG chains of a specific size are often highly preferable over batches having a range of PEG lengths.

The present invention may provide high purity PEG-lipid conjugates having monodisperse PEG chains, and the present invention may also provides methods for the preparation of PEG-lipid conjugates including various lipids such as saturated or unsaturated fatty acids or bile acids. Such PEG-lipid conjugates may be used for drug delivery, especially for intravenous administration of poorly water-soluble agents.

In at least one aspect of the present disclosure, compositions and methods for synthesizing PEG-lipid conjugates comprising a glycerol backbone with either one or two branched monodisperse PEG chains and either one or two lipids groups bonded to the backbone are provided. Spacer or linker groups may be included between the backbone and the PEG chains and/or lipid groups.

In at least one other aspect of the present disclosure, provided are glycerol backbones with two lipids and one monodisperse PEG chain (both isomers), glycerol backbones with one lipid and two monodisperse PEG chain (both isomers), and glycerol backbones with one lipid and one monodisperse PEG chain (all isomers) where the third position on the backbone may be a variety of compounds or moieties.

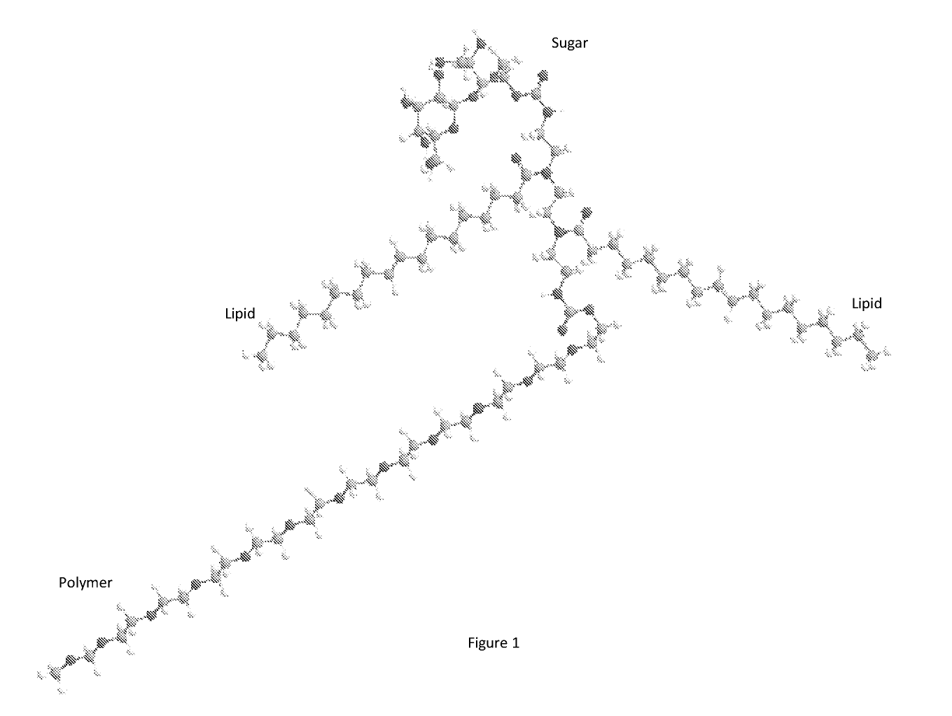

In addition, aspects of the invention may provide methods to make substantially pure 1,2 or 1,3 glycerol isomers. Commercially available 1,2 glycerol lipid diesters may be used to formulate many compounds by linking new moieties to the available position on the glycerol backbone. However, positional transformation may occur during the storage of these 1,2 glycerol diesters resulting in the formation of more stable 1,3 glycerol isomers, which may be present in fractions as great as about 30%. Aspects of the present invention may provide the ability to produce and keep the enantiomer purity of 1, 2 or 1,3 glycerol isomers. While the 1,2 or 1,3 isomers may sometimes be functionally equivalent, the choice of isomer may have implications in a variety of delivery process such as intracellular transport of lipophilic molecules as well as their use as vehicles in pharmaceutical applications. For example, isomers may differ in the ability to stabilize a compound during solubilizing and storage. Chemical Structure 1, shown in FIG. 1, illustrates the difference in steric conformation of two such isomers. FIG. 1 shows Chemical Structure 1 which is a 3D drawing of (1) 1,2-dimyristoyl-glycerol-3-didodecaethylene glycol glycerate and (2) 1,3-dimyristoyl-glycerol-3-didodecaethylene glycol glycerate.

Conjugates having monodisperse PEG chains up to 1200 Daltons may be useful for various drug delivery applications. Conjugates where PEG chains lengths are between about 300 and 700 daltons may be especially useful when formulating liquid dosage-forms such as for intravenous injection or oral solution. Conjugates where PEG chains lengths are between about 600 and 1,200 Daltons may be especially useful for solid dosage forms such as capsules. A combination the above may be useful for making a solid dosage form for poorly water soluble agents in which a liquid form of the above conjugates, typically with PEG chains between about 300 to 700 daltons, is used as a solvent and the solid form of the above conjugates, typically with PEG chains between about 700 to 1,200 Daltons, is used as a solidifier.

The present invention may provide convenient and economical synthesis methods for preparing monodisperse PEG-lipid conjugates and various linear linkage groups for conjugating a lipid to a polymer. The present invention may provide several advantages such as simplified synthesis, high product yield and low starting material costs since commercially available PEG oligomers are extremely expensive and may make them cost prohibitive for large scale production of similar PEG-lipid conjugates. In addition, the present synthesis method may be preferable for preparing a wide range of PEG-spacer-lipid conjugates.

Synthesis of monodisperse PEG chains may involve initially linking a short chain of PEG (having between 1 and 6 subunits) to a protected glycerol backbone. The PEG chain may be lengthened by repeated etherification. An example is shown in Reaction Scheme 1.

By way of example, shown in Reaction Scheme 1, a first reactive PEG oligomer (b) is prepared by protecting (for example, by benzene) a first terminus of a PEG oligomer and creating a reactive second terminus (for example, by a tosyl group as shown). The first reactive oligomer is then combined with a glyceric acid that has a tert-butyl protected carboxylic group (a). The protective group on the glycols is selected to be stable under conditions that remove the protected group on the first terminus of the first reactive oligomer. The reactive second terminus of the oligomer bonds with the two free —OHs of the glycerol to form a di-oligomer glycerol intermediate (b). The protecting groups on the first terminus of the oligomer portion of the intermediate are then removed to expose reactive —OH groups and two reactive PEG oligomers are added to the intermediate to form the extended PEG chains attached to the glycerol backbone (d).

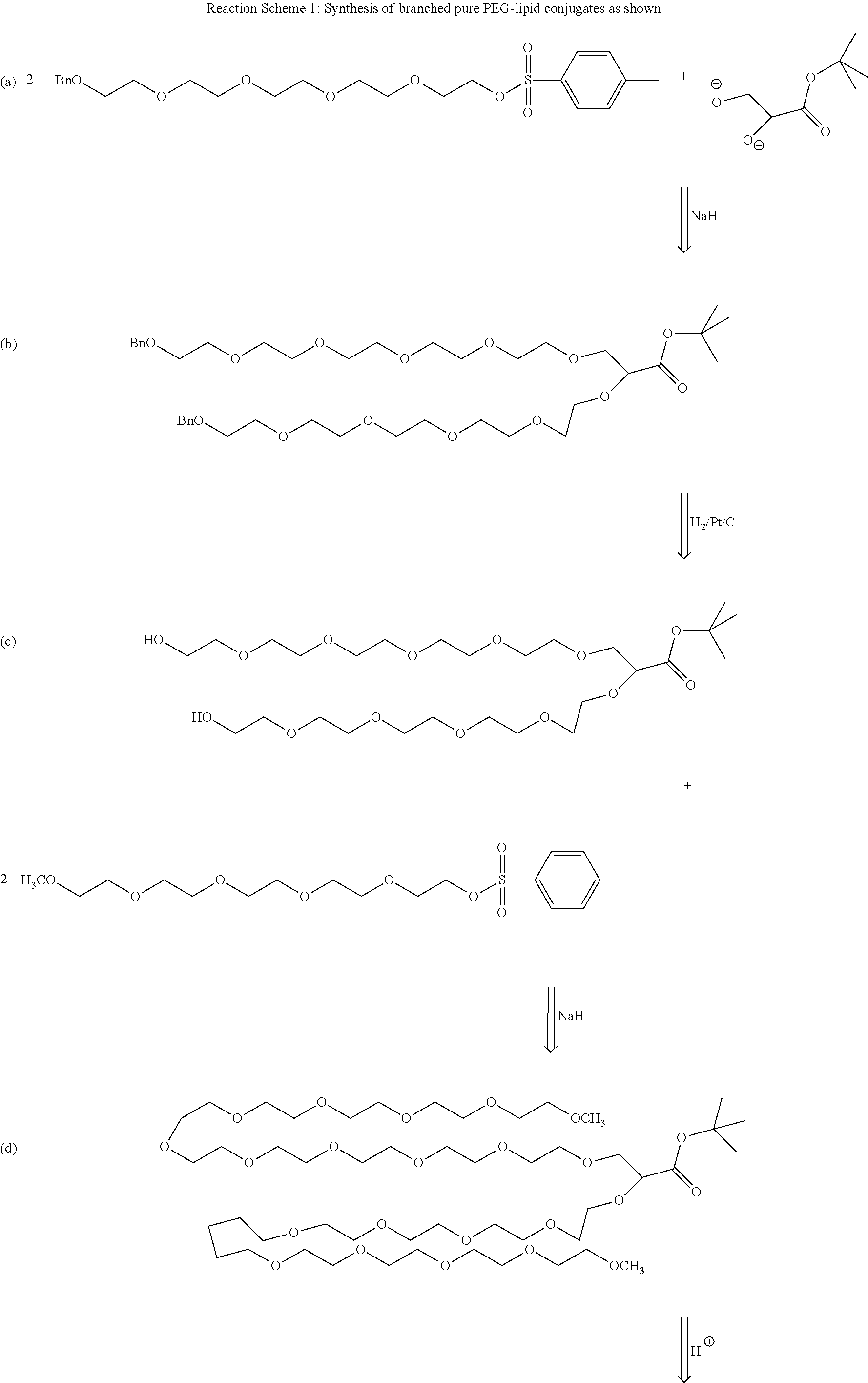

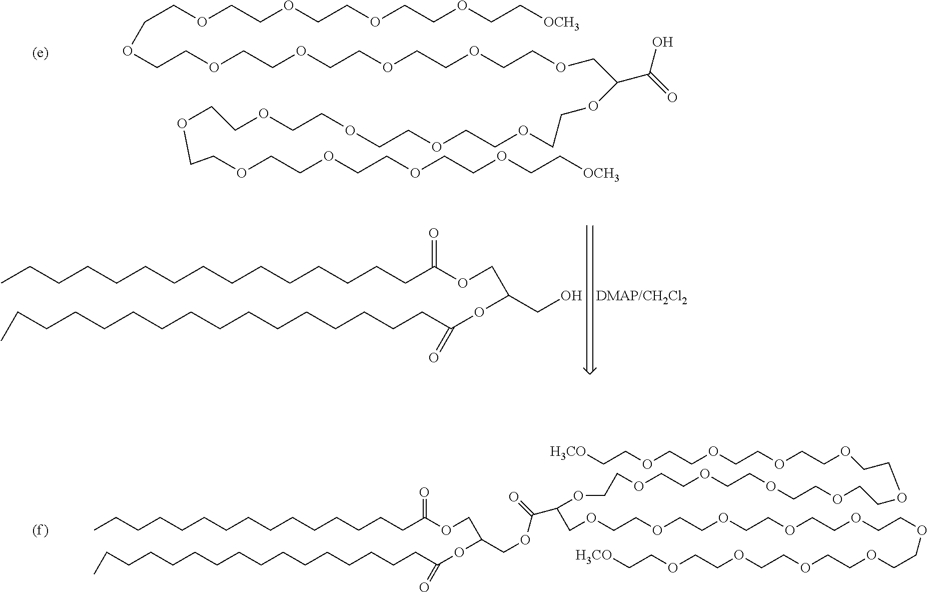

In Reaction Scheme 1, the second reactive PEG oligomer is protected on its first terminus by a terminal methyl group, because a 8 subunit PEG chain is desired. If longer chains are desired, the protective group on the second reactive PEG oligomer is selected to that it may easily be removed for further extension of the PEG chain, for example by using (a) again as the second oligomer. Once the desired chain length is achieved, the protected group of the carboxylic backbone is removed to form the branched PEG chain (e). Intermediate product (e), having a branched monodisperse PEG chain, may then be further reacted to add desired lipids to the glycerol backbone (g). Similarly the synthesis may start with a short PEG chain or prepare the hexaethylene glycol from the etherification of two triethylene glycol or between a triethylene glycol and a monomethoxy triethylene glycol. In this route, two more steps may be involved in the synthesis.

In Reaction Scheme 1, removal of protective benzyl groups to expose a free hydroxyl group may be achieved by any suitable reagents. For example, the benzyl group may be removed by hydrogenation in presence of palladium catalyst before the PEG chain is extended by repeating the etherification process.

Following the synthesis of a PEG chain on a glycerol backbone as exemplified in Reaction Scheme 1, the tert-butyl protecting group may be removed from the backbone, which results in a free carboxylic group. The free carboxylic group may be reacted with a diesters of glycerol in the presence of N,N-dimethylamino pyridine (DMAP) in an inert solvent as shown below in Reaction Scheme 1 (g).

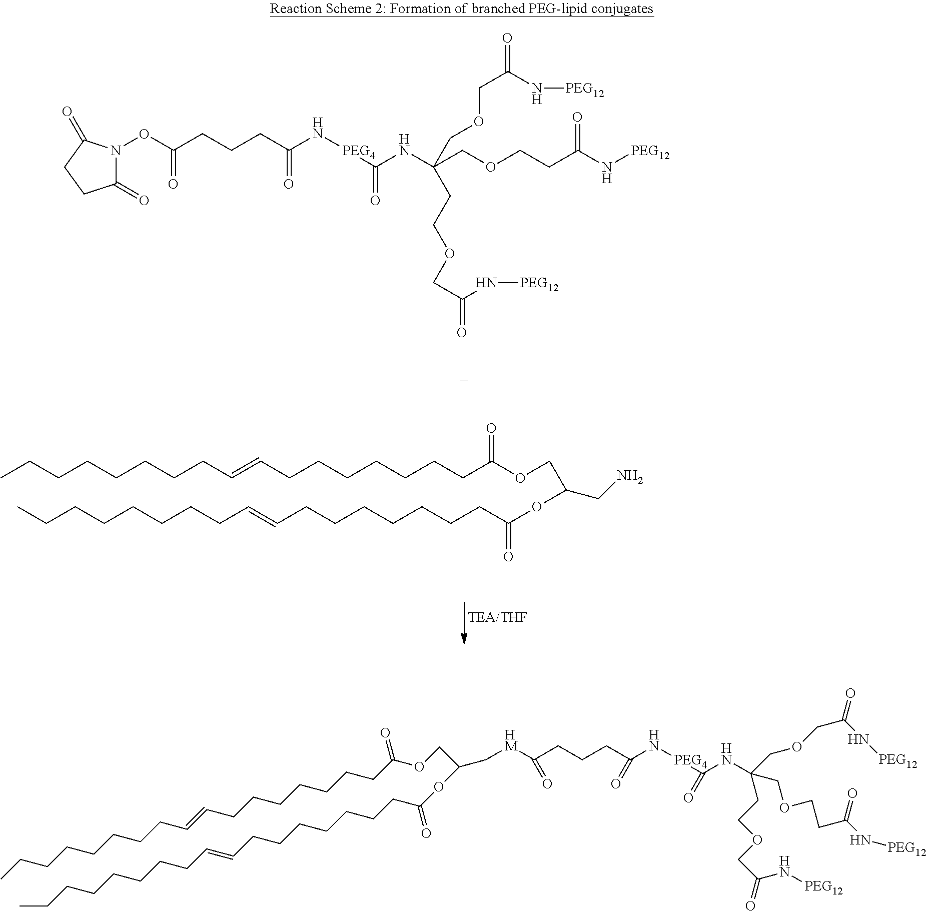

Reaction Scheme 2 depicts an approach to, or an example of, the preparation of a branched PEG-lipid conjugate where a commercially available branched trimethyl and succinimide ester derivative of polyethylene glycol PEG (TMS-(PEG)₁₂) is used in Reaction Scheme 2. In this method, the NHS ester may be spontaneously reactive with the primary amine of the lipid.

While the foregoing illustrates one method to synthesize a particular PEG-lipid conjugate having a single monodisperse PEG chain, the present disclosure more broadly teaches methods and materials to make a wide range of PEG-lipid conjugates.

The first reactive PEG oligomer preferably comprises between 3 to 6 CH₂CH₂O units, and more preferably has 4 to 6 CH₂CH₂O units, though the oligomer may be of any length up to 24 units. Additional reactive oligomers also preferably comprise between 3 to 7 CH₂CH₂O units, and more preferably has 4 to 7 CH₂CH₂O units, though the additional oligomers may be of any length up to 24 units.

The PEG-lipid conjugates of the present invention may each have a branched two or three monodisperse PEG chains. Unless otherwise noted, more than 50% of the PEG chains in a particular conjugate may have the same molecular weight. More preferably, 75% or more have the same molecular weight. Most preferably, more than 90% have the same molecular weight. Also unless otherwise noted, the PEG chains may be comprised of between about 6 and 27 polymer subunits. More preferably the PEG chains are comprised of between about 7 and 27 polymer subunits. Most preferably the PEG chains are comprised of between about 7 and 24 polymer subunits.

In the case of synthesizing 1,2-dimyristoylglycerol-3-diPEG 8-glycerate, the carboxylic group may be protected so that the PEG chains are formed with 2 hydroxyls (see Reaction Scheme 1, compound (a)) It will be appreciated that employing alternate glycerol derivatives as starting components may result in conjugates having PEG chains in different positions. For example, in an aspect where the backbone is selected from glycerol-like analogues or linear amines (tri- or tetra-amines) or amino acids having three available binding sites.

The present invention may be practiced using a wide variety of central backbones. Preferable backbones may have at least three available positions for carbohydrate or lipid or PEG attachments through esterification or etherification. For example, molecules that may be used as the backbone include glycerol or glycerol-like analogues or linear amines or amino acids or triols or diols with a carbonyl or carboxy group or amine, and diamines with a hydroxyl or carbonyl or carboxy group. More preferable the space between the two closest binding positions on the backbone is between 2 to 8 elements such as single carbon or CH2. Most preferable space between the two closest binding positions on the backbone is between 2 and 4 elements.

Examples of glycerol or glyceride or triols or aminodiols and analogues that may be suitable to be used as the central backbone include, but are not limited to, 3-amino-1,2-propanediol, 3-bromo-1,2-propanediol, 3-chloro-1,2-propanediol, 3-fluoro-1,2-propanediol, DL-glyceric acid, diaminopropionic acid, tartaric acid, glucoheptonic acid and 1,2,4-butanetriol, 2,2-Bis(hydroxymethyl)-butyric acid.

Examples of linear amines that may be suitable to be used as the central backbones include, but are not limited to, diethylenetriamine (spermidine), triethylenetriamine (spermine), norspermidine, bis(3-aminopropyl)-1,3-propanediamine, bis(hexamethylene)triamine.

Examples of amino acids with two carboxyl groups or two hydroxyl or two amino groups that may be used as the central backbone may include, but are not limited to, Aspartic Acid, Glutamic Acid, Asparagine, Glutamine, Ornithine, Serine and Threonine, more preferable the amino acids include Aspartic Acid, Glutamic Acid, Ornithine, Serine and Threonine, and most preferable the amino acids include Aspartic Acid, Glutamic Acid, Ornithine and Serine.

If a branched PEG with two chains is desired, a glycerol derivatives as shown in Chemical Structure 2 or Chemical Structure 3 may be used. In these structures, R indicates either a protective group that may be replaced later, or a diacylglycerol that may comprise the final structure. For these conjugates, the PEG chains may be grown in tandem and may be identical in length. Conjugates having two or more PEG chains may be particularly useful as they may function as branched PEG-lipid conjugates with enhanced solubilzing power.

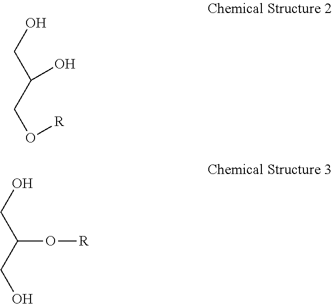

It may be desirable to incorporate linker groups or linkers other than oxy between a diacylglycerol and a branched PEG chain(s). For example, a thiol linker may be employed for applications where a labile bond is useful. Other useful linkers are noted in Table 3 and elsewhere in this specification. For syntheses of conjugates having alternative linkers between the backbone and the PEG chain(s) or diacylglycerol, the linker group may be first attached to a protected glycerol backbone, then the first reactive PEG oligomer may be attached to the free end of the linker and the PEG may be extended as desired. Alternatively, the first reactive PEG oligomer may be attached to the linker before bonding the linker to the backbone. In embodiments with linkers, preferred PEG-reagents may have hydroxyl, amino, carboxyl, isocyanate, thiol, carbonate functional groups. Especially preferred PEG-reagents for use in this embodiment of the inventive method may include PEG-tosylate, PEG-mesylate and succinyl-PEG.

It may be also be desirable to incorporate the same linker groups between the glycerol backbone and the lipid group(s). To obtain such conjugates, either the linker may be bonded with the lipid before attachment to the backbone, or the linker may be bonded to the backbone before attaching the lipid to the linker.

The foregoing approaches describe growing the PEG chain(s) on a backbone that is protected by a removable protecting group. Then, after the PEG is in place, the lipid group or groups are attached to the backbone. However, aspects of the present disclosure may use one lipid as a protecting group on the backbone before growing the PEG chain. This aspect may be especially useful with alkyl chains that don't have reactive groups that need to be protected during PEG attachment and extension. It may be less useful when steroid acid conjugates are desired, as the bile acids tend to have many side groups that may create issues during PEG attachment and extension.

While the synthetic methods described above are useful for making many compounds comprising the invention, in some cases it may be necessary or more convenient to employ other methods. Therefore, aspects of the present disclosure include the more convenient or other methods. For example, if a conjugate having a bile acid and a branched PEG with two 24 subunit PEG chains is desired, such a conjugate may be constructed by synthesizing the monodisperse PEG chains before attaching the bile acid to the central backbone. Similarly, many of the compounds of the invention, including smaller PEGs, may be made by using PEG chains synthesized before attachment to the central backbone. Therefore, aspects of the present disclosure include these disclosed methods and methods known or derived by persons having ordinary skill in the art upon reading the instant disclosure.

Synthesis of other compounds of the invention may also require special considerations. For example, conjugates having linkers between the backbone and diacylglycerol group or a branched PEG chains may preferably be made by building the monodisperse PEG chains before attaching them to the backbone, depending on considerations such as the nature of the bonds in the linkers.

Following the principles described above, a wide variety of PEG-lipid conjugates having two or more monodisperse PEG chains may be synthesized. A number of further specific embodiments are described hereinafter.

Suitable lipids for synthesis of PEG-lipid conjugates may include bile acids (steroid acids) as well as alkyl chains. Therefore, the present invention includes a variety of PEG-lipid conjugates prepared by the present liquid phase synthesis method. The steroid acid-PEG conjugates may, for example, be incorporated into liposomes as a targeting moiety for lipid-based drugs to specific cells or as self-emulsifying drug delivery systems (SEDDS).

Bile acids (steroid acids) constitute a large family of molecules, composed of a steroid structure with four rings, a five or eight carbon side-chain terminating in a carboxylic acid, and the presence and orientation of different numbers of hydroxyl groups. The four rings are labeled from left to right A, B, C, and D, with the D-ring being smaller by one carbon than the other three. An exemplary bile acid is shown in Chemical Structure 4. All bile acids have side chains. A carboxyl group may be amide-linked with taurine or glycine and the nuclear hydroxyl groups may be esterified with glucuronide or sulfate, which may be essential for the formation of water soluble bile salts from bile alcohols, where R₁ and R₂ may be hydroxyl or proton.

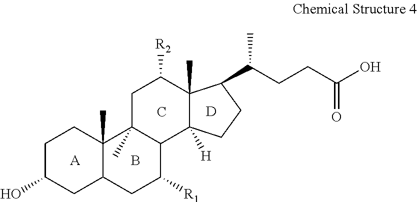

Currently only a few modifications in structure have been studied with respect to the physical-chemical properties of bile salts. For example, patent publication (WO 02083147) discloses bile salt fatty acid conjugate in which a bile acid or bile salt is conjugated in position 24 (carboxyl) with a suitable amino acid, and the unsaturated C═C bond is conjugated with one or two fatty acid radicals having 14-22 carbon atoms. That conjugate is intended to be used as a pharmaceutical composition for the reduction of cholesterol in blood, for the treatment of fatty liver, hyperglycemia and diabetes. Another patent publication (US 2003212051) discloses acyclovir-bile acid prodrugs in which a linker group may be used between the bile acid and the compound.

In one general embodiment or aspect, the present invention may provide PEG-lipid conjugates according to general Formula I. The difference between the two aspects shown in Formula I is the relative position of the polymer and lipid chains along the glycerol backbone.

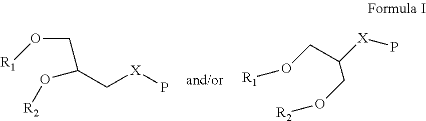

There are several alternative embodiments or aspects of Formula I. In at least one aspect of Formula I, R1 and R2 may the same or different and are selected from the saturated and/or unsaturated alkyl groups listed in Table 1 or Table 2; X is —O—C(O)—, —O—, —S—, —NH—C(O)— or a linker selected from Table 3; and P is a branched PEG chain.

In at least one other aspect of Formula I, one of R1 and R2 is an alkyl group and the other is H. In these aspects of Formula I, at least one of R1 or R2 may be a saturated or unsaturated alkyl group having between 6 and 22 carbon atoms. In a preferred aspect, R1 and R2 are the same and include between 6 and 22 carbon atoms and more preferably between 12 and 18 carbon atoms. The terms “alkyl” encompasses saturated or unsaturated fatty acids.

The present invention also provides PEG-lipid conjugates according to general formula II.

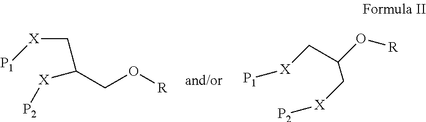

Again, there are several alternative aspects or embodiments of Formula II. In one at least one aspect of Formula II, R is a dialkylglycerol consisting of fatty acid chains listed in Tables 1 or Table 2; X is —O—C(O)—, —O—, —S—, —NH—C(O)— or a linker selected from Table 3; and P1 and P2 are the same branched PEG chains. By providing two branched PEG chains, conjugates according to Formula II may provide advantages over conjugates having a single longer PEG chain.

TABLE 1
Saturated lipids for use in the invention:

| | | | | Melting |
|---|---|---|---|---|
| Common | | | | point |
| name | IUPAC name | Chemical structure | Abbr. | (° C.) |
| Butyric | Butanoic acid | CH₃(CH₂)₂COOH | C4:0 | −8 |
| Caproic | Hexanoic acid | CH₃(CH₂)₄COOH | C6:0 | −3 |
| Caprylic | Octanoic acid | CH₃(CH₂)₆COOH | C8:0 | 16-17 |
| Capric | Decanoic acid | CH₃(CH₂)₈COOH | C10:0 | 31 |
| Lauric | Dodecanoic acid | CH₃(CH₂)₁₀COOH | C12:0 | 44-46 |
| Myristic | Tetradecanoic acid | CH₃(CH₂)₁₂COOH | C14:0 | 58.8 |
| Palmitic | Hexadecanoic acid | CH₃(CH₂)₁₄COOH | C16:0 | 63-64 |
| Stearic | Octadecanoic acid | CH₃(CH₂)₁₆COOH | C18:0 | 69.9 |
| Arachidic | Eicosanoic acid | CH₃(CH₂)₁₈COOH | C20:0 | 75.5 |
| Behenic | Docosanoic acid | CH₃(CH₂)₂₀COOH | C22:0 | 74-78 |

TABLE 2
Unsaturated lipids for use in the invention:

| | | Δ^{x} | |
|---|---|---|---|
| | | Location of | # carbon/ |
| Name | Chemical structure | double bond | double bonds |
| Myristoleic acid | CH₃(CH₂)₃CH═CH(CH₂)₇COOH | cis-Δ⁹ | 14:1 |
| Palmitoleic acid | CH₃(CH₂)₅CH═CH(CH₂)₇COOH | cis-Δ⁹ | 16:1 |
| Oleic acid | CH₃(CH₂)₇CH═CH(CH₂)₇COOH | cis-Δ⁹ | 18:1 |
| Linoleic acid | CH₃(CH₂)₄CH═CHCH₂CH═CH(CH₂)₇COOH | cis,cis-Δ⁹,Δ¹² | 18:2 |
| α-Linolenic acid | CH₃CH₂CH═CHCH₂CH═CHCH₂CH═CH(CH₂)₇COOH | cis,cis,cis- | 18:3 |
| | | Δ⁹,Δ¹²,Δ¹⁵ | |
| Arachidonic acid | CH₃(CH₂)₄CH═CHCH₂CH═CHCH₂CH═CHCH₂CH═CH(CH₂)₃COOH^{NIST} | cis,cis,cis,cis- | 20:4 |
| | | Δ⁵Δ⁸,Δ¹¹,Δ¹⁴ | |
| Erucic acid | CH₃(CH₂)₇CH═CH(CH₂)₁₁COOH | cis-Δ¹³ | 22:1 |

TABLE 3
Additional Linkers for use in the invention

| No | Symbol | Linker |
|---|---|---|
| 1 | N₁ | 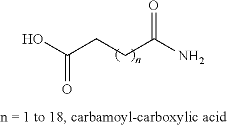 |
| 2 | N₂ | 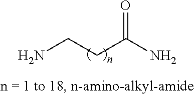 |
| 3 | N₃ | 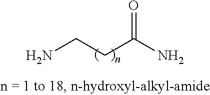 |
| 7 | N₇ | 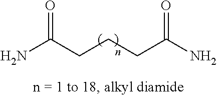 |
| 8 | N₈ | 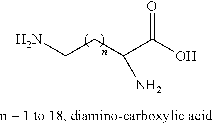 |
| 9 | N₉ | 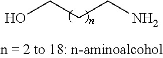 |
| 10 | N₁₀ | 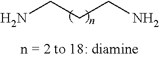 |
| 11 | N₁₁ | 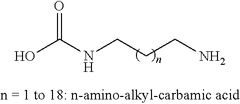 |
| 12 | N₁₂ | 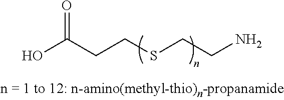 |
| 13 | S₁ | 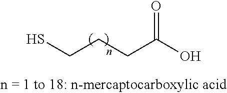 |
| 14 | S₂ | 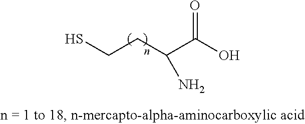 |
| 15 | S₃ | 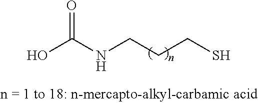 |
| 16 | S₄ | 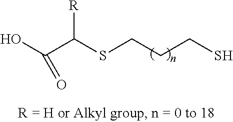 |
| 17 | S₅ | 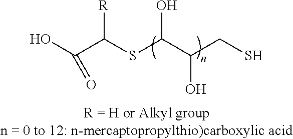 |
| 18 | S₆ | 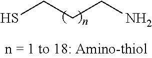 |
| 19 | S₇ | 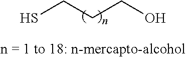 |
| 20 | S₈ | 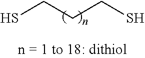 |
| 21 | S₉ | 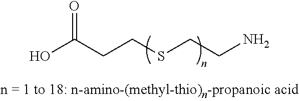 |
| 22 | Ac₁ | 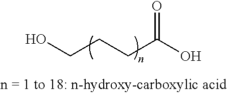 |
| 23 | Ac₂ | 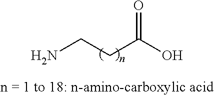 |
| 24 | Ac₃ | 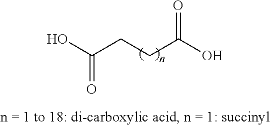 |
| 25 | Ac₄ | 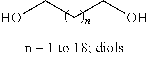 |
| 26 | Ac₅ | 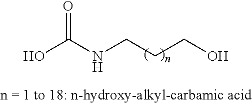 |
| 27 | Ac₆ | 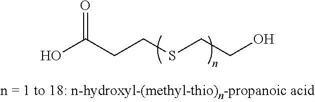 |

PEG-lipid conjugates of the present invention also include compounds where the lipid portion comprises one or two bile acids. These conjugates may have the same structures as shown in Formula I and Formula II, except that the alkyl groups may be replaced by bile acids. For bile acid conjugates, aspects and preferred embodiments are the same as described for the PEG-alkyl conjugates. Because bile acids are similarly lipophilic to alkyl groups, bile acid conjugates also share similar physical properties and may be generally suitable for some of the same uses as PEG-alkyl conjugates.

Chemical Structure 5 shows two aspects of the present invention having a branched PEG chains and a lipid consisting of two bile acids attached to a glycerol backbone.

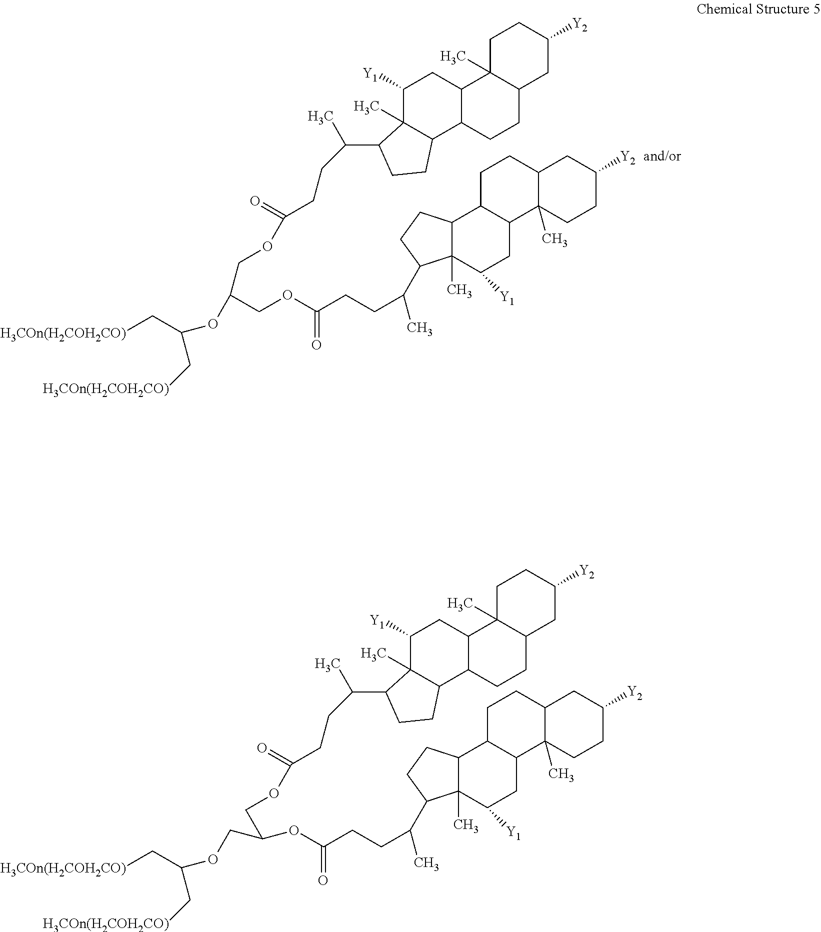

In Chemical Structure 5, Y₁ and Y₂ may be the same or different and are OH or H or CH₃, or are selected to accord with the bile acids shown in Table 4. Similarly, bile acids with differing side chains (as shown in Table 4) may be conjugated to the glycerol backbone. Table 4 lists bile acid and its derivatives that may be useful in practicing the present invention.

TABLE 4
Bile acid (steroid acid) and its analogues for use in the Invention

| Name | Chemical Structure | Other Name |
|---|---|---|
| Cholic acid | 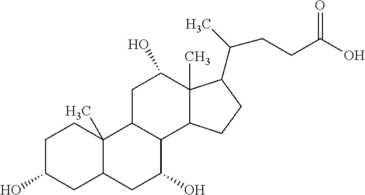 | 3α,7α,12α-trihydroxy-5β- cholanoic acid |
| Desoxycholic acid | 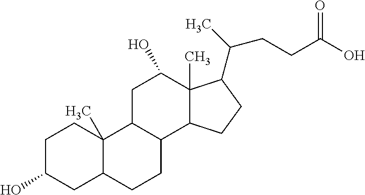 | 3α,12α-Dihydroxy-5β- cholanic acid |
| 5-Cholenic acid-3β-ol | 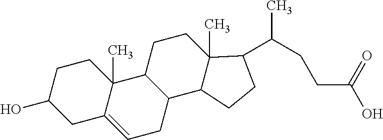 | 3β-Hydroxy-5-cholen-24- oic acid |
| Dehydrocholic acid | 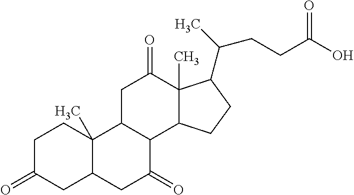 | 3,7,12-Trioxo-5β-cholanic acid |
| Glycocholic acid | 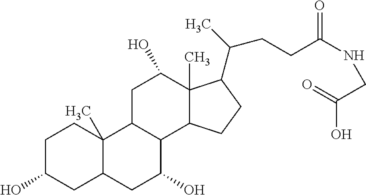 | N-(3α,7α,12α-Trihydroxy- 24-oxocholan-24-yl)- glycine |
| Glycodeoxycholic acid | 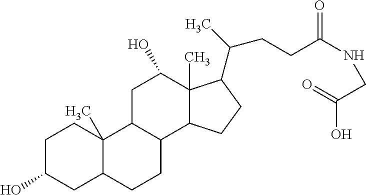 | N-(3α,12α-Dihydroxy-24- oxocholan-24-yl)glycine |
| Chenodeoxycholic acid | 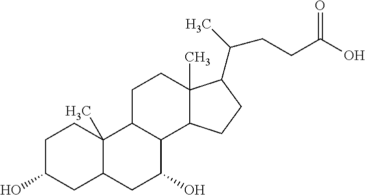 | 3α,7α-dihydroxy-5β- cholanic acid |
| Glycochenodeoxycholic acid | 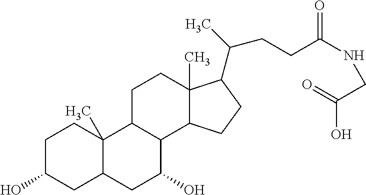 | N-(3α,7α-Dihydroxy-24- oxocholan-24-yl)glycine |
| Ursodeoxycholic acid | 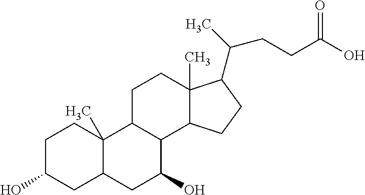 | Ursodiol |
| Lithocholic acid | 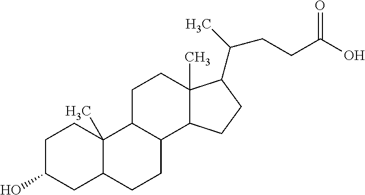 | 3α-Hydroxy-5β-cholan- 24-oic acid |
| Hyodeoxycholic acid | 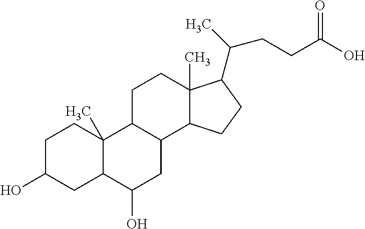 | 3α,6α-Dihydroxy-5β- cholan-24-oic acid |
| 5β-Cholanic acid-3,7-dione | 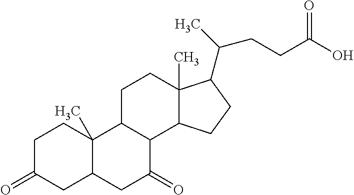 | 3,7-Diketo-5β-cholan-24- oic acid |

Yet another aspect of the invention includes compounds according to Formula I where either R1 or R2 is a bile acid and the other is an alkyl group. An example of this aspect of lipid polymer conjugate is shown in Chemical Structure 6.

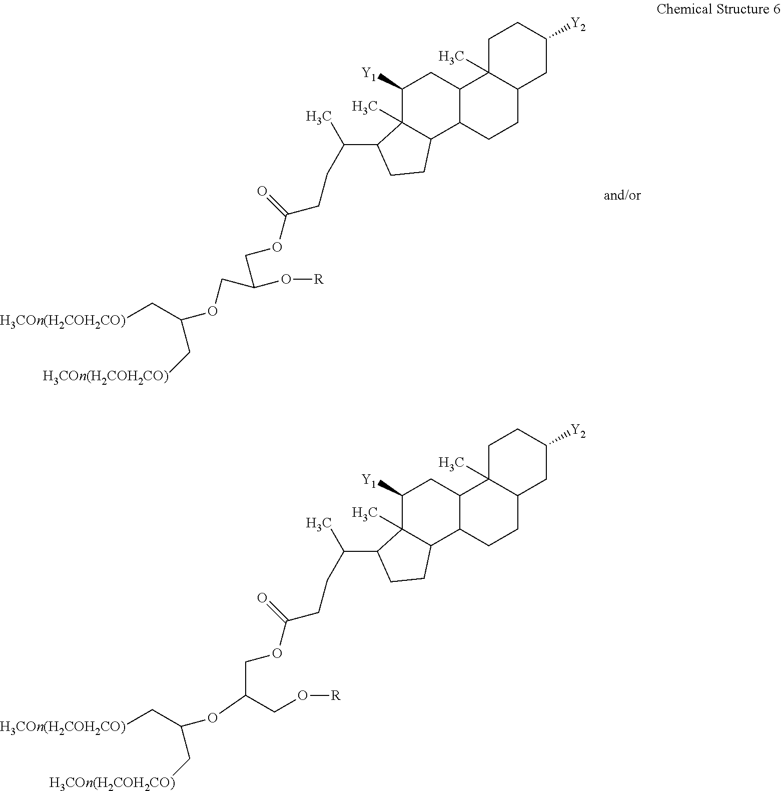

In Chemical Structure 6, Y1 and Y2 are the same or different and are OH or H or CH3 or selected in accord with the bile acids shown in Table 4. Also, the side chain of the bile acid may be varied according to the structures shown in Table 4. R is saturated and/or unsaturated alkyl group selected from Tables 1 and Table 2.

Another preferred embodiment for the compound of general Formula II is a PEG-bile acid conjugate according to Chemical Structure 7.

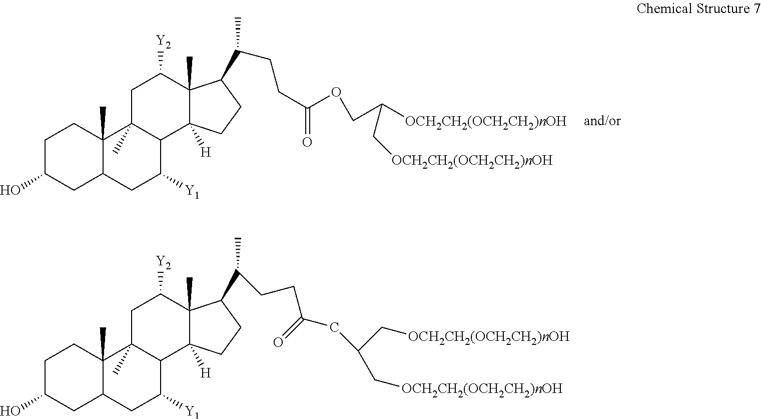

In Chemical Structure 7, Y₁ and Y₂ are OH or H or CH₃ or selected according to the bile acids shown in Table 4. Also, the side chain of the bile acid may be varied according to the structures shown in Table 4.

Another further preferred embodiment for the compound of general Formula II is a PEG-cholesterol conjugate according to Chemical Structure 8.

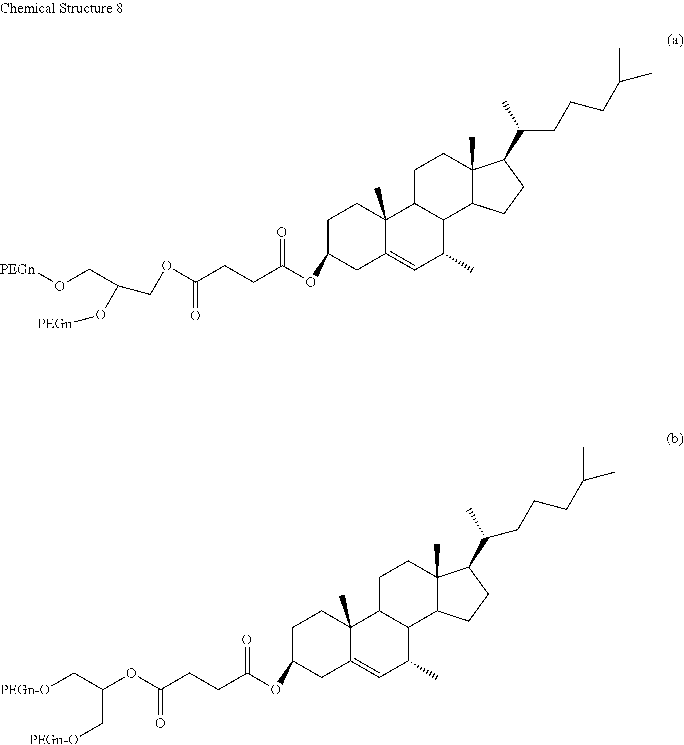

Another embodiment of the present invention is represented in Reaction Scheme 3. In this method, any suitable amino acid, such as lysine, is reacted with mPEG-NHS ester compounds in the presence of -Triethylamine (TEA) in Tetrahydrofuran (THF) to form a branched PEG and produce the final product of 1,2-dimyristoyl-rac-3-dimPEG 12-lysinate. It will be appreciated that monodisperse PEG chains of many discrete lengths may be used.

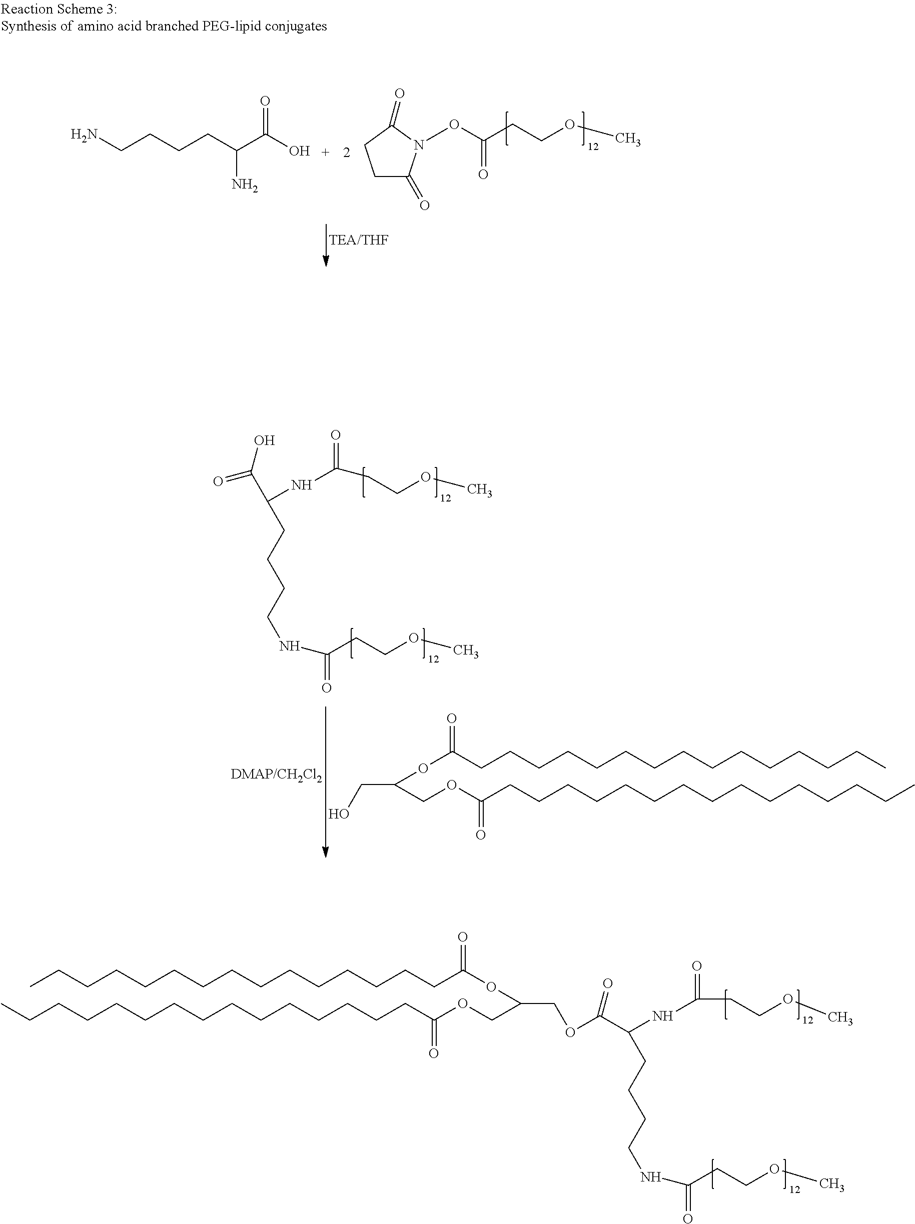

Another embodiment of the present invention, represented in Reaction Scheme 4, involves reaction of DL-1,2-isopropylideneglycerol intermediate with fatty acid to give I or with cholesterol to give II, respectively. Removal of ispropyl groups by any desired methods provides intermediate products III and IV respectively.

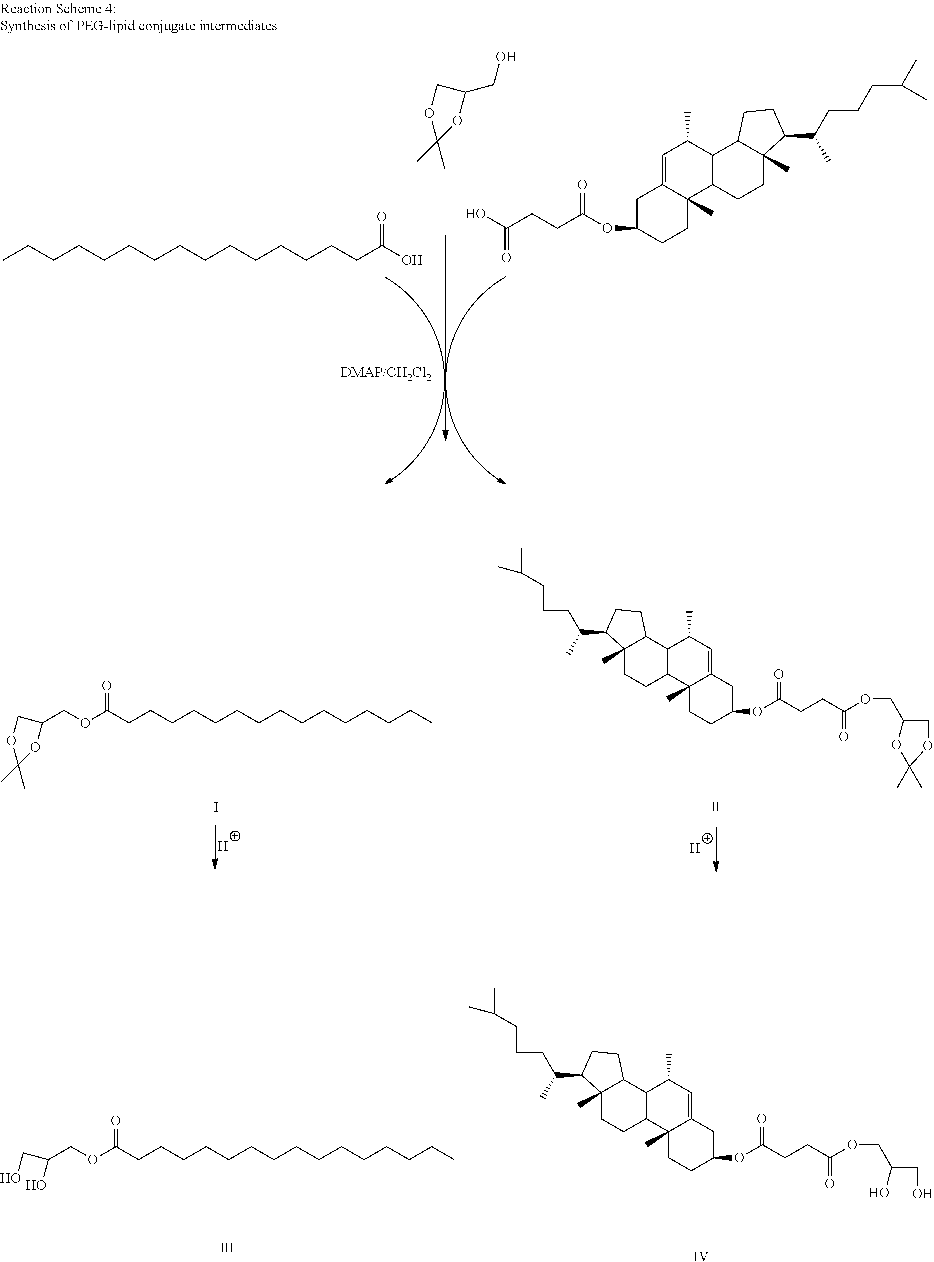

The described methods may be used to prepare a variety of novel PEG-lipid conjugates. For example, the methods may be used to prepare 3-PEG-1,2-alkylglycerol in pure form containing any fatty acid chain. Preferred fatty acids range from carbon chain lengths of about C6 to C22, preferably between about 10 and about C18.

The described methods may be used to prepare a variety of novel PEG-lipid conjugates. For example, the methods may be used to prepare 3-PEG-1,2-disteroid acid-glycerol in pure form containing any bile acid chain.

The described methods may be used to prepare a variety of novel branched PEG-lipid conjugates. For example, the methods may be used to prepare 3-alkylgl-1,2-bisPEG-gycerol in pure form containing any fatty acid chain. Preferred fatty acids range from carbon chain lengths of about C6 to C22, preferably between about C10 and about C18 (Reaction Scheme 5).

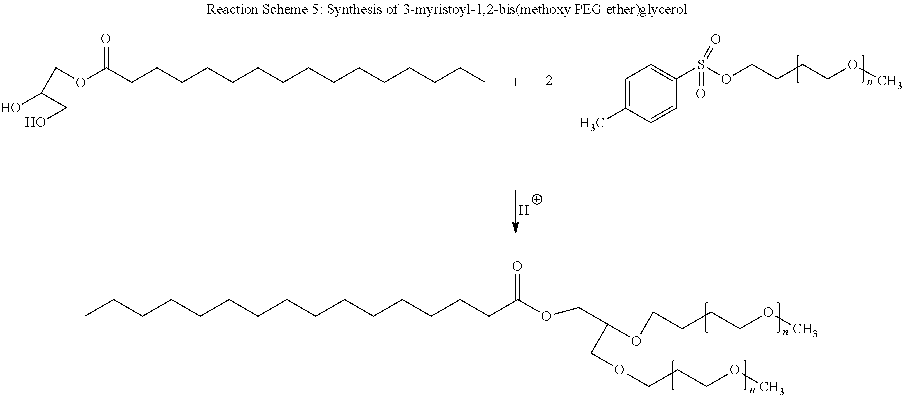

Reaction Scheme 5 results in a compound having a glycerol backbone, an lipid group, and two monodisperse PEG chains. However, it is to be understood that extending the PEG chain as exemplified in Reaction Scheme 1 may be done with other oligomers such as triethylene glycols or between triethylene glycol and monotriethylene glycol as previously described.

The described methods may be used to prepare a variety of novel branched PEG-lipid conjugates. For example, the methods may be used to prepare 3-steroid acid-1,2-bisPEG-gycerol in pure form containing steroid acid-glycerol in pure form containing any bile acid chain (Reaction Scheme 6).

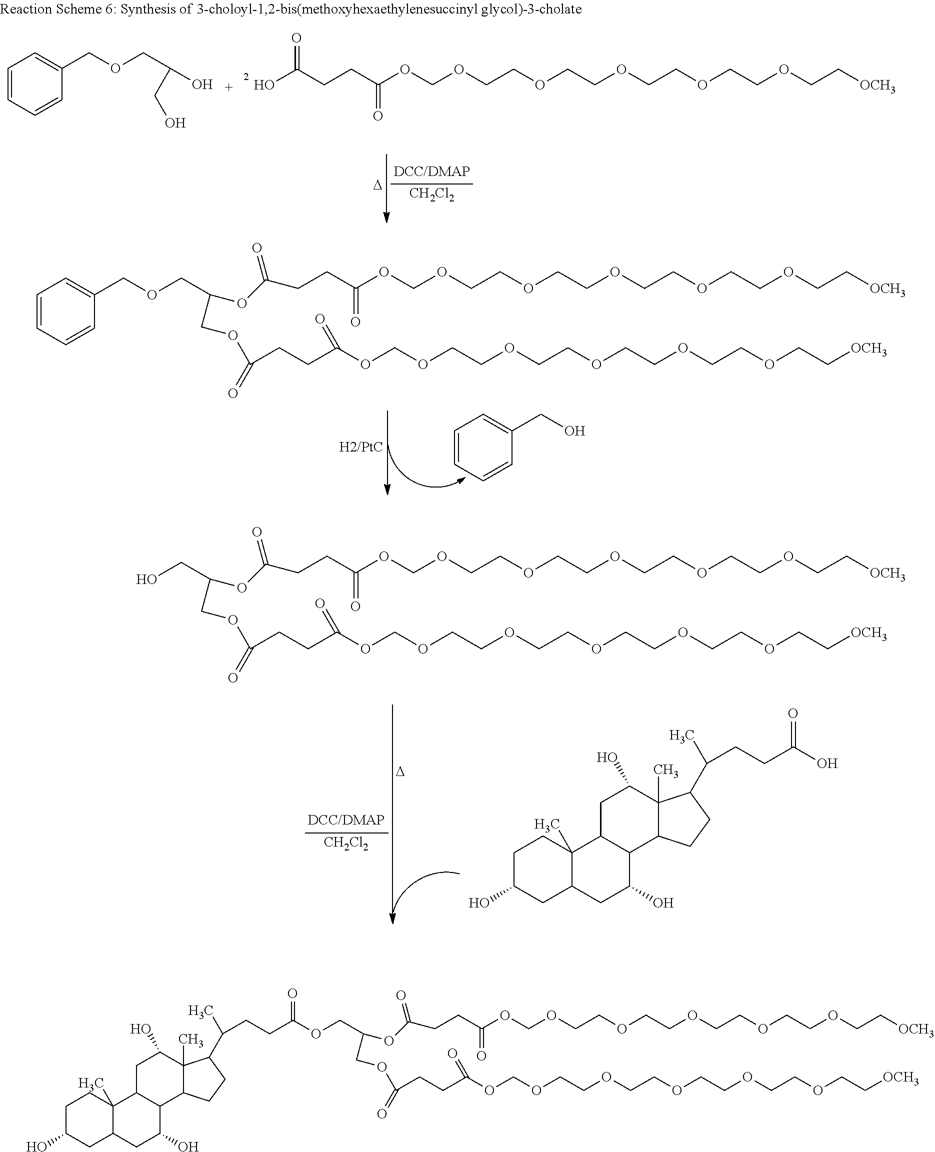

One preferred use for the inventive PEG-lipid is in the preparation of liposomes and other lipid-containing formulations. In accordance with the present invention, a pharmaceutical composition may include one or more genetic vectors, antisense molecules, proteins, peptides, bioactive lipids or drugs. For example, the active agent may include one or more drugs (such as one or more anticancer drugs or other anticancer agents). Typically hydrophilic active agents may be added directly to the formulation and hydrophobic active agents may be dissolved by PEG-lipid before mixing with the other ingredients.

Suitable active agents that may be present in the inventive formulation include one or more genetic vectors, antisense molecules, proteins, peptides, bioactive lipids or drugs, such as are described above. The inventive PEG-lipid may be used to administer active agents that may be safer in presence of PEG oligomer and may be suitable for intravenous use.

Preferred active agents which may be compatible with the present invention include agents which act on the peripheral nerves, adrenergic receptors, cholinergic receptors, the skeletal muscles, the cardiovascular system, smooth muscles, the blood circulatory system, synaptic sites, neuroeffector junctional sites, endocrine and hormone systems, the immunological system, the reproductive system, the skeletal system, the alimentary and excretory systems, the histamine system and the central nervous system. Suitable agents may be selected from, for example, proteins, enzymes, hormones, nucleotides, polynucleotides, nucleoproteins, polysaccharides, glycoproteins, lipoproteins, polypeptides, steroids, terpenoids, retinoids, anti-ulcer H2 receptor antagonists, antiulcer drugs, hypocalcemic agents, moisturizers, cosmetics, etc. Active agents may be analgesics, anesthetics, anti-arrythmic agents, antibiotics, antiallergic agents, antifungal agents, anticancer agents (e.g., mitoxantrone, taxanes, paclitaxel, camptothecin, and camptothecin derivatives (e.g., SN-38), gemcitabine, anthacyclines, antisense oligonucleotides, antibodies, cytoxines, immunotoxins, etc.), antihypertensive agents (e.g., dihydropyridines, antidepressants, cox-2 inhibitors), anticoagulants, antidepressants, antidiabetic agents, anti-epilepsy agents, anti-inflammatory corticosteroids, agents for treating Alzheimers or Parkinson's disease, antiulcer agents, anti-protozoal agents, anxiolytics, thyroids, anti-thyroids, antivirals, anoretics, bisphosphonates, cardiac inotropic agents, cardiovascular agents, corticosteroids, diuretics, dopaminergic agents, gastrointestinal agents, hemostatics, hypercholesterol agents, antihypertensive agents, immunosuppressive agents, anti-gout agents, anti-malarials, anti-migraine agents, antimuscarinic agents, anti-inflammatory agents, such as agents for treating rheumatology, arthritis, psoriasis, inflammatory bowel disease, Crohn's disease, or agents for treating demyelinating diseases including multiple sclerosis, ophthalmic agents; vaccines (e.g., against influenza virus, pneumonia, hepatitis A, hepatitis B, hepatitis C, cholera toxin B-subunit, typhoid, plasmodium falciparum, diptheria, tetanus, herpes simplex virus, tuberculosis, HIV, bordetela pertusis, measles, mumps, rubella, bacterial toxoids, vaccinea virus, adenovirus, SARS virus, canary virus, bacillus calmette Guerin, klebsiella pneumonia vaccine, etc.), histamine receptor antagonists, hypnotics, kidney protective agents, lipid regulating agents, muscle relaxants, neuroleptics, neurotropic agents, opioid agonists and antagonists, parasympathomimetics, protease inhibitors, prostglandins, sedatives, sex hormones (e.g., androgens, estrogens, etc.), stimulants, sympathomimetics, vasodilators and xanthins and synthetic analogs of these species. The therapeutic agents may be nephrotoxic, such as cyclosporins and amphotericin B, or cardiotoxic, such as amphotericin B and paclitaxel, etopside, cytokines, ribozymes, interferons, oligonucleotides, siRNAs, RNAis and functional derivatives of the foregoing.

Chemotherapeutic agents may be well suited for use in the inventive method. The inventive PEG-lipid formulations containing chemotherapeutic agents may be injected directly into the tumor tissue for delivery of the chemotherapeutic agent directly to cancer cells. In some cases, particularly after resection of a tumor, the liposome formulation may be implanted directly into the resulting cavity or may be applied to the remaining tissue as a coating.

The branched PEG-lipid in present invention may be used for preparing various dosage forms including tablets, capsules, pills, granules, suppositories, solutions, suspensions and emulsions, pastes, ointments, gels, creams, lotions, eye drop, powders and sprays in addition to suitable water-soluble or water-insoluble excipients.

The inventive PEG-lipid conjugates may be used to deliver the active agent to targeted cells in vivo. For example, the composition may be delivered orally, by injection (e.g., intravenously, subcutaneously, intramuscularly, parenterally, intraperitoneally, by direct injection into tumors or sites in need of treatment, etc.), by inhalation, by mucosal delivery, locally, and/or rectally or by such methods as are known or developed. Formulations containing PEGylated cardiolipin may also be administered topically, e.g., as a cream, skin ointment, dry skin softener, moisturizer, etc.

For in vivo use, the present disclosure provides the use of a composition as herein described containing one or more active agents for preparing a medicament for the treatment of a disease. In other words, the disclosure provides a method of using a composition as herein described, containing one or more active agents, for treating a disease. Typically, the disease is present in a human or animal patient. In a preferred embodiment, the disease is cancer, in which instance, the inventive composition comprises one or more anticancer agents as active agents. For example, in accordance with the invention, the compositions as described herein may be employed alone or adjunctively with other treatments (e.g., chemotherapy or radiotherapy) to treat cancers such as those of the head, neck, brain, blood, breast, lung, pancreas, bone, spleen, bladder, prostate, testes, colon, kidney, ovary and skin. The compositions of the present invention, comprising one or more anticancer agents, may be especially preferred for treating leukemias, such as acute leukemia (e.g., acute lymphocytic leukemia or acute myelocytic leukemia). Kaposi's sarcoma also may be treated using the compositions and methods of the present invention.

The following structures further illustrating the present invention.

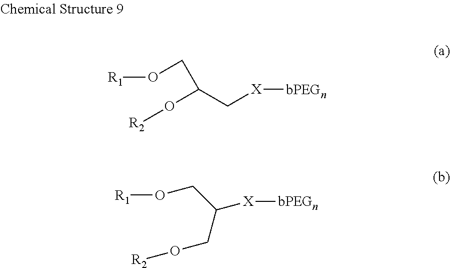

In Chemical Structure 9 “X” is a linker such as oxy, thiol, amino, —COO—, —OCOO—, succinyl, haloid and those listed in Table 3. Where bPEG represents a branched PEG and “n” is the number of repeating units. These structures represent intermediates in growing a branched PEG with two or more monodisperse PEG chains on a central backbone, n may be generally between about 6 and 24. The PEG chain may be extended through a sequential etherification starting with smaller chain such as triethylene glycol or tetraethylene glycol directly attached to the glycerol via a linker. The terminal group on the PEG chain may be, but is not limited to, a methyl group. Here R₁ and R₂ may be the same or difference fatty acids and may be selected from Tables 1 and 2 or steroid acids selecting from Table 4.

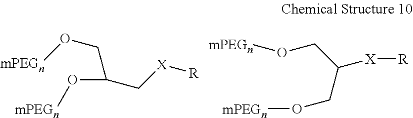

In Chemical Structure 10 “X” is the linker including oxy, thiol, amino, —COO—, —OCOO—, succinyl, haloid and those shown in Table 3. “n” is the number of repeating units. These structures represent the final step in growing two monodisperse PEG chains on a glycerol backbone. The “R” is a lipid such as dialkylglycerol group containing saturated (Table 1) or unsaturated fatty acids (Table 2) or cholyl groups or analogs (Table 4). Terminal groups besides methyl may be included on the PEG chains.

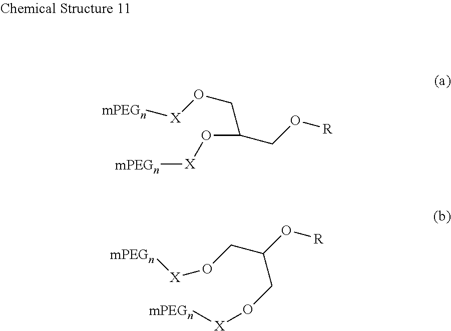

In Chemical Structure 11 “X” is the linker such as oxy, thiol, amino, —COO—, —OCOO—, succinyl, haloid and alike and those shown in Table 3. “n” is the number of repeating units. These structures represent the final step in growing two monodisperse PEG chains on a glycerol backbone. Similarly the PEG chain may be extended through a sequential etherification starting with smaller chain such as triethylene glycol or tetraethylene glycol directly attached to the glycerol via a linker. The “R” is lipid such as a dialkylglycerol group containing saturated (Table 1) or unsaturated fatty acids (Table 2) or cholyl groups or analogs (Table 4). Terminal groups besides methyl may be included on the PEG chains.

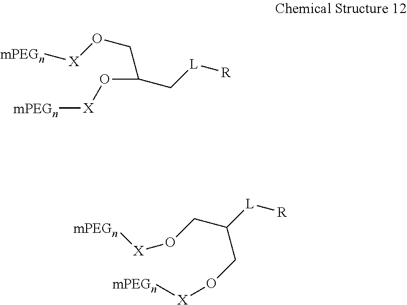

In Chemical Structure 12 “X” and “L” are the same or different linkers such as oxy, thiol, amino, —COO—, —OCOO—, succinyl, haloid and those shown in Table 3. “n” is the number of repeating units. These structures represent the final step in growing two monodisperse PEG chains on a glycerol backbone, so n may generally be between about 5 and 24. The “R” is a lipid such as a dialkylglycerol group containing saturated (Table 1) or unsaturated fatty acids (Table 2) or cholyl groups and its analogs (Table 4). Terminal groups besides methyl may be included on the PEG chains.

Embodiments of the present invention are described herein in the context of preparation of pharmaceutical compositions including purified branched PEG-lipid conjugates for increasing the solubility and enhancing the delivery of active agents. The approximate preferable compositions for formulated drug products are generally described herein, though different drugs typically have differing optimal formulations.

For IV solutions, the preferable concentration of drug is 0.1% to 30%. More preferable is 1 to 10%. Most preferable is 1 to 5%. The preferable ratio of PEG-lipid to the drug (PEG-Lipid/drug) is 1 to 20. More preferable is 1 to 10. Most preferable is 1 to 5.

For oral solutions, the preferable concentration of drug is 1% to 40%. More preferable is 2.5 to 30%. Most preferable is 5 to 30%. The preferable ratio of PEG-lipid to the drug (PEG-Lipid/drug) is 0.5 to 20. More preferable is 1 to 5. Most preferable is 1 to 3.

For ophthalmic preparations, the preferable concentration of drug is 0.01 to 5%. More preferable is 0.05 to 2%. Most preferable is 0.1 to 2%. The preferable ratio of PEG-lipid to the drug (PEG-Lipid/drug) is 1 to 20. More preferable is 3 to 15. Most preferable is 5 to 10.

For topical solutions, the preferable concentration of drug is 0.05 to 5%. More preferable is 0.1 to 5%. Most preferable is 0.1 to 2%. The preferable ratio of PEG-lipid to the drug (PEG-Lipid/drug) is 1 to 20. More preferable is 3 to 15. Most preferable is 5 to 10.

For oral capsules, the preferable capsule content of drug is 10 mg to 250 mg. More preferable is 25 mg to 200 mg. Most preferable is 25 mg to 100 mg. The preferable ratio of PEG-lipid to the drug (PEG-Lipid/drug) is 1 to 10. More preferable is 1 to 5. Most preferable is 2 to 5.

For topical preparations, the preferable concentration of drug is 0.05 to 5%. More preferable is 0.1 to 5%. Most preferable is 0.5 to 2%. The preferable ratio of PEG-lipid to the drug (PEG-Lipid/drug) is 1 to 50. More preferable is 3 to 20. Most preferable is 5 to 10.

While the foregoing discussion has focused on polymer-lipid conjugates having a glycerol backbone and including a PEG chains, the invention further includes alternate backbones and polymers. For example, glycerol or glyceride or triols or aminodiols and analogues may be suitable to be used as the central backbone including, and not limited to, 3-amino-1,2-propanediol, 3-bromo-1,2-propanediol, 3-chloro-1,2-propanediol, 3-fluoro-1,2-propanediol, DL-glyceric acid, aspartic acid, glutamic acid,1,2,4-butanetriol and 2,2-Bis(hydroxymethyl)-butyric acid may be used as alternative backbones to synthesize similar PEG-lipid conjugates. Linear amines may be suitable to be used as the central backbones including, and not limited to, diethylenetriamine, spermidine, norspermidine, bis(3-aminopropyl)-1,3-propanediamine, bis(hexamethylene)-triamine. Amino acids with two carboxyl groups or two hydroxyl or two amino groups may be used as the central backbone, preferable amino acids are Aspartic Acid, Glutamic Acid, Glutamine, Asparagine, Serine and Threonine, more preferable are Aspartic Acid, Glutamic Acid, Serine and Threonine, and most preferable are Aspartic Acid, Glutamic Acid and Serine. Chemical Structure 13 illustrates a conjugate of the invention employing aspartic acid as a backbone. To prepare this conjugate, the starting material may be oleoyl alcohol instead of oleic acid since there are two carboxyl groups in the amino acid already. A succinate linker has been used to attach the PEG to the backbone. In such alternative embodiments, the PEG chain (or alternative polymer chain) is always monodisperse.

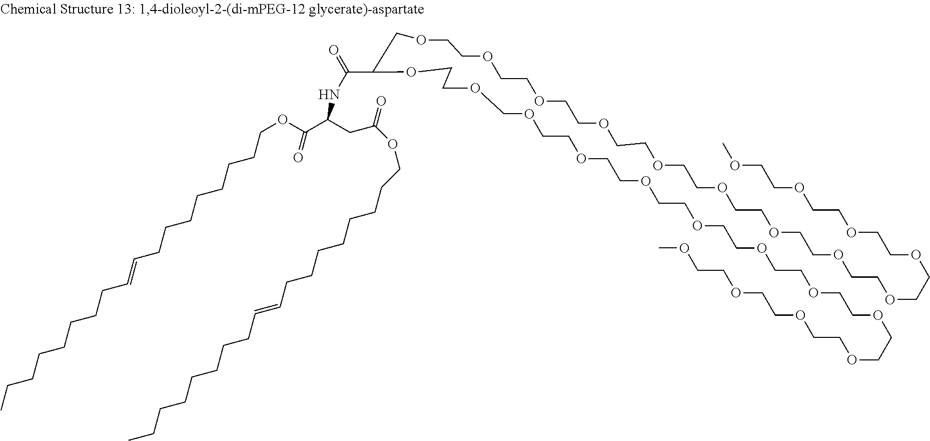

Propylene glycol and methylene glycol oligomers may be used as alternatives to ethylene glycol oligomers. Also, it is possible to create copolymers or block copolymers of these basic building blocks.

The synthetic methods described herein may be modified in any suitable manner. For example, the PEG-reagents for use in the inventive method may be any PEG derivative, which is capable of reacting with hydroxyl or amino group of central glycerol or 3-amino-1,2-propanediol group or like or functional group of most any linker.

The solvent for PEG-lipid conjugation reaction in the inventive method includes most any solvent such as a polar aprotic solvent. For example, N,N-dimethylformamide (DMF), dimethylsulfoxide (DMSO), pyridine, tetrahydrofuran (THF), dichloromethane, chloroform, 1,2-dichloroethane, dioxane and the like may be as solvents for PEG-lipid conjugation.

In one aspect, the invention is a method of making a PEG chain of a defined length, the method comprises: (a) selecting a glycerol derivative with a glycerol protecting group that is stable under a first set of conditions and convertible to free hydroxyl groups under a second set of conditions; (b) selecting an initial PEG oligomer having between 1 and 12 subunits, where the initial PEG oligomer has an oligomer protecting group on its first terminus and the oligomer protecting group converts to a hydroxyl group under the first set of conditions, and where the initial PEG oligomer has a reactive group on its second terminus, the reactive group forming a bond with a compound having a free hydroxyl group; (c) reacting the glycerol derivative with the initial PEG oligomer to form a glycerol-PEG conjugate; (d) removing the oligomer protective group by exposing the conjugate to the first set of conditions; (e) repeating steps (f), (g) and (h) between 0 and 6 additional times, where steps are as described below; (f) selecting an extending PEG oligomer having between 2 and 11 subunits, where the extending PEG oligomer has an oligomer protecting group on its first terminus and the oligomer protecting group converts to a hydroxyl group under the first set of conditions, and where the extending PEG oligomer has a reactive group on its second terminus, the reactive group forming a bond with a compound having a free hydroxyl group; (g) reacting the glycerol-PEG conjugate with the extending PEG oligomer to form an extended glycerol-PEG conjugate; (h) removing the oligomer protective group by exposing the conjugate to the first set of conditions; (i) terminating the PEG chain by either step (j) or steps (k) and (1), where the steps are as described below; (j) adding a terminal group to the free hydroxyl group of the extended glycerol-PEG conjugate; or (k) selecting a terminal PEG oligomer having between 2 and 11 subunits, where the terminal PEG oligomer has terminal group on its first terminus, and where the terminal PEG oligomer has a reactive group on its second terminus, the reactive group forming a bond with a compound having a free hydroxyl group; and (l) reacting the glycerol-PEG conjugate or extended glycerol-PEG conjugate with the terminal PEG oligomer; and (m) exposing the terminated glycerol-PEG conjugate to the second set of conditions. The terminal group may be a methyl group. The first set of conditions may be catalytic reduction. The second set of conditions may be exposure to acid. The glycerol derivative may be a compound represented by the formula shown at Reaction Scheme 1(a). The glycerol derivative may be a compound represented by the formula shown as Chemical Structure 2. The glycerol derivative may be a compound represented by the formula shown as Chemical Structure 3. The glycerol derivative may be a compound represented by the formula shown as Chemical Structure 4. The glycerol protecting group may be an alkyl group. The method may further comprise the steps of: (n) removing the glycerol protecting group; and (o) bonding a lipid group to the glycerol backbone.

In another aspect, a chemical composition is provided wherein the chemical composition includes a PEG-lipid conjugate, the PEG-lipid conjugate comprises: a glycerol backbone; a lipid group covalently attached to the glycerol backbone; and a PEG chain covalently attached to the glycerol backbone, where the PEG chain has a MW between about 200 and 1200 Daltons, and where greater than about 75 percent of the PEG chains of the conjugate molecules in the composition have the same MW. Greater than about 90 percent of the PEG chains of the conjugate molecules in the composition may have the same MW. The PEG chain may have a MW greater than about 550 Daltons. The lipid may be an alkyl group. The alkyl group may be selected from the alkyl groups in Table 1 and Table 2. The composition may further comprise a second lipid covalently attached to the glycerol backbone. The second lipid may be an alkyl group. The second alkyl group may be selected from the alkyl groups in Table 1 and Table 2. The lipid may be a bile acid. The bile acid may be selected from the bile acids in Table 4. The bile acid may be cholesterol. The composition may further comprise a linker group between the glycerol backbone and the PEG chain. The linker may be selected from the group consisting of —S—, —O—, —N—, —OCOO—, and the linkers in Table 3. The composition may further comprise a second PEG chain covalently attached to the glycerol backbone. The linkage between the glycerol backbone and the second PEG chain may be selected from a group consisting of —O—C(O)—, —O—, —S—, and —NH—C(O)—. The linkage between the glycerol backbone and the second PEG chain may be selected from Table 3.

In another aspect, chemical compositions according to paragraph 088 are provided, where the glycerol backbone is replaced by a backbone selected from the group consisting of 3-amino-1,2-propanediol, 3-bromo-1,2-propanediol, 3-chloro-1,2-propanediol, 3-fluoro-1,2-propanediol, DL-glyceric acid, aspartic acid, glutamic acid, and 1,2,4-butanetriol.

In another aspect, chemical compositions according to claim paragraph 088 are provided, where the PEG chains are replaced by polymers selected from the group consisting of polymethylene glycol, polypropylene glycol, and copolymers comprised of a at least two of the monomers selected from the group consisting of methylene glycol, propylene glycol and ethylene glycol.

In another aspect, the following compounds are provided: the compound represented by the formula shown at Reaction Scheme 1 (a); the compound represented by the formula shown as Chemical Structure 2; the compound represented by the formula shown as Chemical Structure 3; the compound represented by the formula shown as Chemical Structure 4; the molecules of 1,2-isopropylidene-glycerol-3-ethylene glycol, 1,2-isopropylidene-glycerol-3-diethylene glycol, 1,2-isopropylidene-glycerol-3-triethylene glycol, 1,2-isopropylidene-glycerol-3-tetraethylene glycol, 1,2-isopropylidene-glycerol-3-pentaethylene glycol and 1,2-isopropylidene-glycerol-3-hexaethylene glycol, 1,2-isopropylidene-glycerol-3-heptaethylene glycol and 1,2-isopropylidene-glycerol-3-octaethylene glycol; and the molecules of 1,3-diacylglycerol-2-ethylene glycol, 1,3-diacylglycerol-2-diethylene glycol, 1,3-diacylglycerol-2-triethylene glycol, 1,3-diacylglycerol-2-tetraethylene glycol, 1,3-diacylglycerol-2-pentaethylene glycol, 1,3-diacylglycerol-2-hexaethylene glycol, 1,3-diacylglycerol-2-heptaethylene glycol and 1,3-diacylglycerol-2-octaethylene glycol.

In another aspect, the invention includes a method for increasing the bioavailability and/or solubility of an active agent, the method comprising: formulating the active agent with one or more of the a PEG-lipid conjugates of the present invention and administering the PEG-lipid conjugate based formulation to an animal or human.

In another aspect, a chemical compound having the formula is provided:

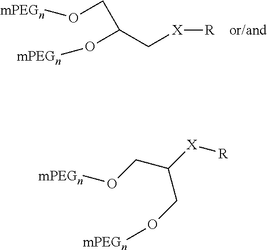

where n is between about 3 and 24; R is a lipid; and where X is a linker group. X may have a MW between about 14 and 620. X may be selected from the group consisting of oxy, thiol, amino, —COO—, —OCOO—, succinyl, haloid and linkers shown in Table 3. n may be between about 4 and 16. More preferably, n may be between about 7 and 16. The terminus of the PEG chain may have a MW between about 15 and 250. The terminus of the PEG chain may be a methyl group. R may be a dialkylglycerol consisting of fatty acids selected from Table 1 or Table 2. R may be a diester consisting of two bile acids selected from Table 4 and a central backbone of glycerol. R may contain cholesterols.

In another aspect, a chemical compound having the formula is provided:

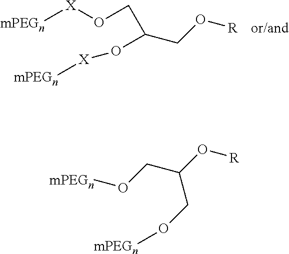

where n is between about 3 and 24; R is a lipid; R is a lipid; and where X are the same or different linker groups. X may have a MW between about 14 and 620. X may be selected from the group consisting of oxy, thiol, amino, —COO—, —OCOO—, succinyl, haloid and linkers shown in Table 3. n may be between about 4 and 24. n is preferably between about 7 and 24. The terminus of the PEG chain may have a MW between about 15 and 250. The terminus of the PEG chain may be a methyl group. R may be a dialkylglycerol consisting of fatty acids selected from Table 3 or Table 4. R may be a diester consisting of bile acids selected from Table 4. R may contain cholesterols.

In another aspect, a chemical compound having the formula is provided:

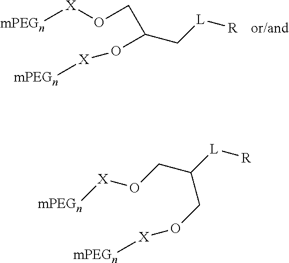

where is between about 3 and 24; R is a lipid; R is a lipid; L is a linker group; and where X are the same or different linker groups. X may have a MW between about 14 and 620. X may be selected from the group consisting of oxy, thiol, amino, —COO—, —OCOO—, succinyl, haloid and linkers shown in Table 3. n may be between about 4 and 24. n is preferable between about 7 and 24. The termini of the PEG chains may have a MW between about 15 and 250. The termini of the PEG chains may be methyl groups. R may be a dialkylglycerol consisting of fatty acids selected from Table 1 or Table 2. R may be a diester consisting of bile acids selected from Table 4. R may contain cholesterols. X may b selected from the group consisting of oxy, thiol, amino, —COO—, —OCOO—, succinyl, haloid and linkers shown in Table 3.

### EXAMPLES

The following examples are further illustrating the invention and should not be constructed as in any way limiting its scope.

### Example 1

### Synthesis of 3-Oleoyl-1,2-bis(methoxyhexathylene glycol)glycerol

### Part 1A: 3-Benzyl-1,2-bis(methoxyhexathylene glycol)glycerol

To a three-necked flask, (±)-3-Benzyloxy-1,2-propanediol (1.2 g, 6 mmol), NaH (0.96 g, 40 mmol) and dry THF (150 mL) were added. A dry THF solution (50 mL) of monomethoxyhexaethylene glycol tosylate (5.4 g, 12 mmol) was then added to the mixture drop-wise at room temperature. The mixture was refluxed for 24 hours and cooled to room temperature. Ice-cold methanol was added to the reaction mixture to quench excessive NaH. The solvent was evaporated and the crude product was extracted with 5% HCl (w/v) and CH₂Cl₂. The solvent was evaporated and further purified by gel permeation chromatography to yield 85% of colorless liquid.

### Part 1B: 3-hydroxyl-1,2-bis(methoxyhexaethylene glycol)glycerol

To a solution of 5 grams of 3-Benzyl-1,2-bis(methoxyhexaethylene glycol)glycerol in 20 mL of n-Hexane, 5 drops of acetic acid and 0.6 g of palladium black were added. The mixture was purged with pure hydrogen at 30° C. in atmosphere for approximately 60 minutes to remove the benzyl protection group on the 3′-hydroxy. After the phenol was replaced by hydrogen, the solution was cooled to 4 to 6° C. and the catalyst was removed by filtration. Solvent was evaporated to yield 98% of the final product.

### Part 1C: 1,2-dioleoylglycerol-3-(1,2-bis(methoxyhexaethylene glycol)glycerate)

6.5 g of the product from 1B (10 mmoles), 6.8 g of dioleoylglycerol (11 mmoles), 9.6 g of N,N′-Dicyclohexylcarbodiimide (50 mmol) and a catalytic amount of DMAP (0.6 g, 5 mmoles) in anhydrous CH₂Cl₂ (400 mL) was stirred at 25° C. for 12 h under nitrogen, after which the N, N′-dicyclohexylurea salts were precipitated and removed by filtration. The filtrates were evaporated under reduced pressure to yield approximately 60% of the final product shown by Chemical Structure 14.

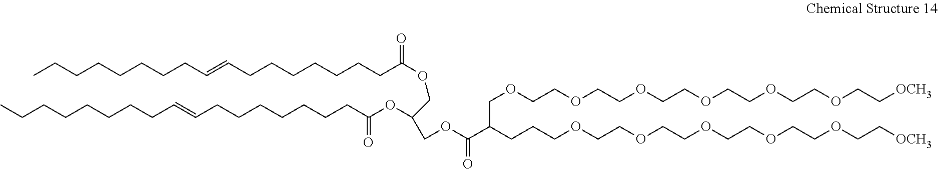

### Example 2

### Synthesis of 1,2-dioleoyl-rac-3-monomethoxydodecaethylene glycol (mPEG-12)-glycerol

The general steps for this synthesis are showed in Reaction Scheme 7.

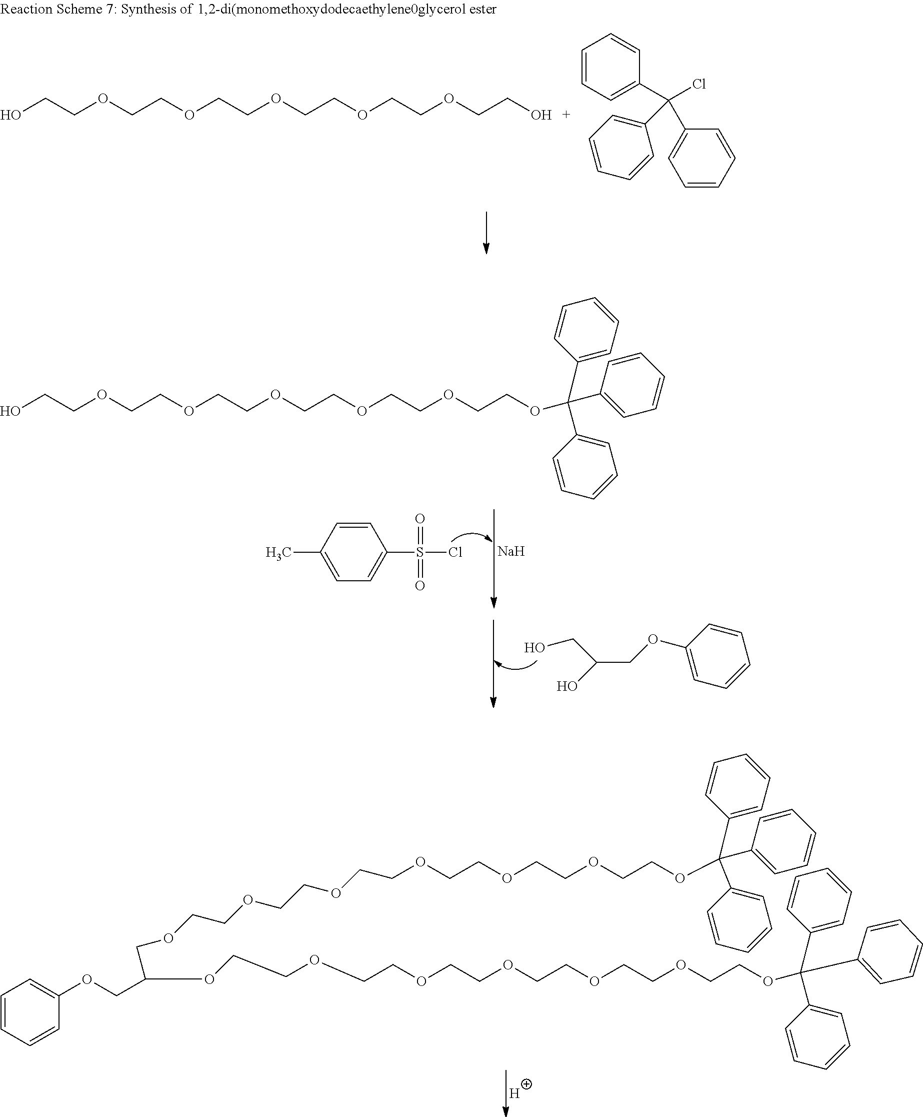

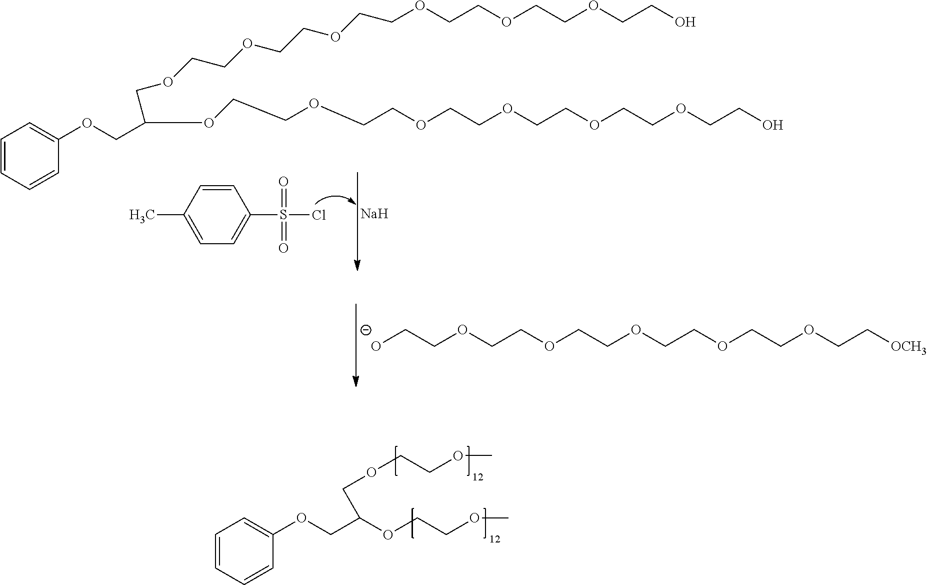

0.1 moles of hexaethylene glycol was mixed with 0.15 moles of pyridine and heated to 45-50° C. and 0.1 moles of trityl (Tr) chloride was added. The reaction was carried over night (approximately 16 hours) under constant stifling and then cooled down to room temperature and extracted with toluene. The extract was washed with water, then extracted with hexane and dried over MgSO₄. The solvent was removed under vacuum, a light yellow oily Tr-hexaethylene glycol was obtained (yield 70 to 85%).

0.1 moles of Tr-hexaethylene glycol and 0.101 moles of p-toluenesulfonyl chloride were mixed in 100 mL of methylene chloride. The homogeneous mixture was cooled to 0° C. in a dry-ice-acetone bath and 45 g of KOH was added in small portions under vigorous stirring while maintaining the reaction temperature below 5° C. The reaction was completed under constant stirring for 3 hours at 0° C. The crude product was diluted with 100 mL of methylene chloride, then 120 mL of ice-cold water was added. The organic layer was collected, and the aqueous phase was extracted with methylene chloride (2×50 mL). The combined organic layers were washed with water (100 mL) and dried over MgSO₄. The solvent was removed under vacuum to yield (87 to 99%) clear oil.

To a three-necked flask, 3-phenoxypropane-1,2-diol (0.05 mol) and NaH (0.2 mol) and dry THF (200 mL) were charged. A dry THF solution (125 mL) of Tr-hexaethylene glycol tosylate (0.1 mol) was added to the mixture dropwise at room temperature. The mixture was refluxed for 24 hours, and cooled to room temperature. Ice-cold methanol was added to the reaction mixture to quench excessive NaH. The solvent was evaporated and the crude product was extracted with 5% HCl (w/v) and CH₂Cl₂. The crude product was not purified further but taken directly to the next stage of synthesis.

The trityl protecting group was removed by stirring 10 g of 1,2-dihexaethylene glycol-3-phenylglycerol for 3 hours in acidic methanol solution (180 mL MeOH:20 mL, 1 N HCl). The mixture was neutralized with sodium hydrogen carbonate and extracted in chloroform (3×150 mL) and dried over sodium sulfate. Filtration and evaporation of the solvent yields the product (75-80%) of 1,2-dihexaethylene glycol-3-benzylglycerol (Chemical Structure 15).

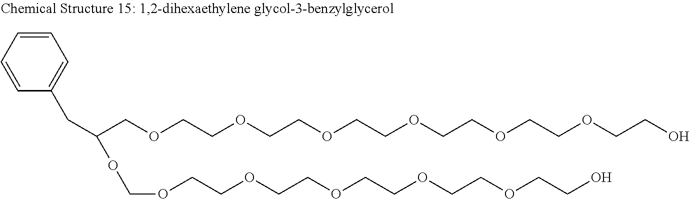

In a three-necked flask, 1,2-dihexaethylene glycol-3-benzylglycerol (0.05 mol), NaH (0.4 mol) and dry THF (500 mL) were mixed. A dry THF solution (200 mL) of monomethoxyl-hexaethylene glycol tosylate (0.11 mmol) was added to the mixture dropwise at room temperature. The mixture was refluxed for 24 hours, and then cooled to room temperature. Ice-cold methanol was added to the reaction mixture to quench excessive NaH. The solvent was evaporated and the crude product was extracted with 5% HCl (w/v) and CH₂Cl₂. The solvent was evaporated and further purified by gel permeation chromatography to yield 83% of 1,2-dimonomethoxydodecaethylene glycol-3-benzylglycerol.

The above crude product was transferred to a high pressure resistant glass flask and 200 mL of dry methylene chloride and 10% palladium on carbon (1.5 g). Hydrogenolysis was carried out by purging pure hydrogen at 30° C. in atmosphere for approximately 60 minutes to remove the benzyl protection group on the 3′-hydroxy. After the phenol was replaced by hydrogen, the solution was cooled to 4 to 6° C. and the catalyst was removed by filtration. Solvent was evaporated to yield 95 to 98% of the final product.

In the above PEG chain extension reaction, the starting PEG reagent preferably comprises 1 to 6 CH₂CH₂O unit, and more preferably has 3 to 6 CH₂CH₂O unit, and more preferably has 4 to 6 CH₂CH₂O units. The reaction between glycerol and the PEG-reagent may occur in the presence or the absence of a linker group. Preferred PEG-reagents have hydroxyl, amino, carboxyl, isocyanate, thiol, carbonate functional groups. Especially preferred PEG-reagents for use in this embodiment of the inventive method include succinyl-PEG, trityle-PEG, PEG-mesylate and PEG-tosylate. Following the reaction between the glycerol and the PEG-reagent, the protecting group at the 3′-hydroxyl is removed.

0.1 moles of 1,2-dimonomethoxydodecaethylene glycol-3-hydroxyl-glycerol was constantly stirred under nitrogen in 250 mL of chloroform. 0.1 mole of 1,2-dioleoylglycerate was dissolved with 250 mL of chloroform and added to this heterogeneous mixture of dihydroxyacetone and followed by adding 19.2 g of N,N′-Dicyclohexylcarbodiimide (0.1 mol) and a catalytic amount of DMAP (1.2 g, 0.01 moles) in anhydrous CH₂Cl₂ (total volume of 400 mL) was stirred at 25° C. for 12 h under nitrogen, after which the N,N′-dicyclohexylurea salts were precipitated and removed by filtration. The filtrates were evaporated under reduced pressure to yield approximately 60% of the final product shown by Chemical Structure 16.

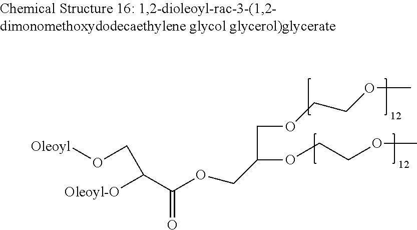

### Example 3

### Synthesis of 1,3-dioleoyl-rac-2-monomethoxyDodecaethylene glycol (mPEG-12)-glycerol

The general steps for this synthesis is showed in the following scheme (Reaction Scheme 8):

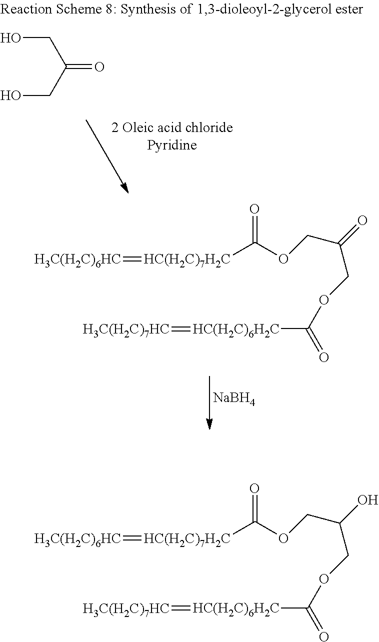

0.033 moles of dihydroxyacetone was constantly stirred under nitrogen in 150 mL of chloroform. 0.06 mole of oleoyl chloride was dissolved with 150 mL of chloroform and added to this heterogeneous mixture of dihydroxyacetone and followed by adding 10 mL of anhydrous pyridine. The reaction proceeded for 30 minutes under constant stirring at room temperature. The mixture turned homogeneous and the reaction was completed when no detectable oleoyl chloride was in the mixture. The bulk solvent was removed under vacuum. The residue was wash with water then extracted with ethyl acetate. The aqueous phase was repeatedly extracted with ethyl acetate and the organic layers were combined and washed again with water, dried over sodium sulfate and evaporated. The resulting oily product was recrystallized from methanol to give 3-(octadec-10-enoyloxy)-2-oxopropyl octadec-9-enoate (% of yields 75-80) with a melting temperature of 43-44°.

The 1,3-dioleate (0.02 moles) was dissolved with 150 mL of tetrahydrofuran (THF) and 10 mL of water. The heterogeneous solution was chilled to 5° C. in an ice-bath. A solution of sodium borohydride (0.026 mol in THF) was added in small portions. After 30 minutes excess borohydride was destroyed by adding approximately 1 mL of glacial acetic acid, the solution was then diluted with chloroform, and washed with water and dried over magnesium sulfate. An oil was obtained which partially crystallized to needle-like crystals of 2-hydroxy-3-(octadec-10-enoyloxy)propyl octadec-9-enoate (yields 80 to 90%) with a melting temperature of 20-22° C.

From the above intermediate product, 1,3-dioleoyl-rac-glyecrol-rac-2-(dimonomethoxy-dodecaethylene glycol (mPEG-12)-glycerol) (Chemical Structure 17) was prepared after the reaction and work-up as described in the Examples 1 and 2.

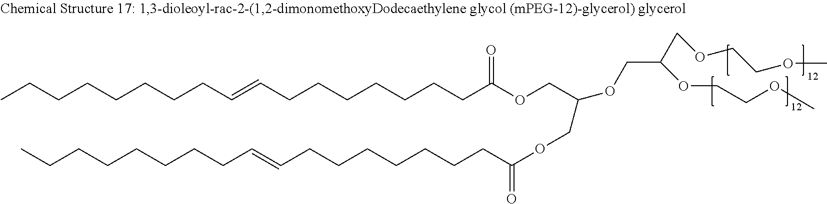

### Example 4

### 1,2-dimyristoyl-rac-3-dodecapropylene glycol (PPG-12)-glycerol

The general steps for this synthesis is showed in the following scheme (Reaction Scheme 9):

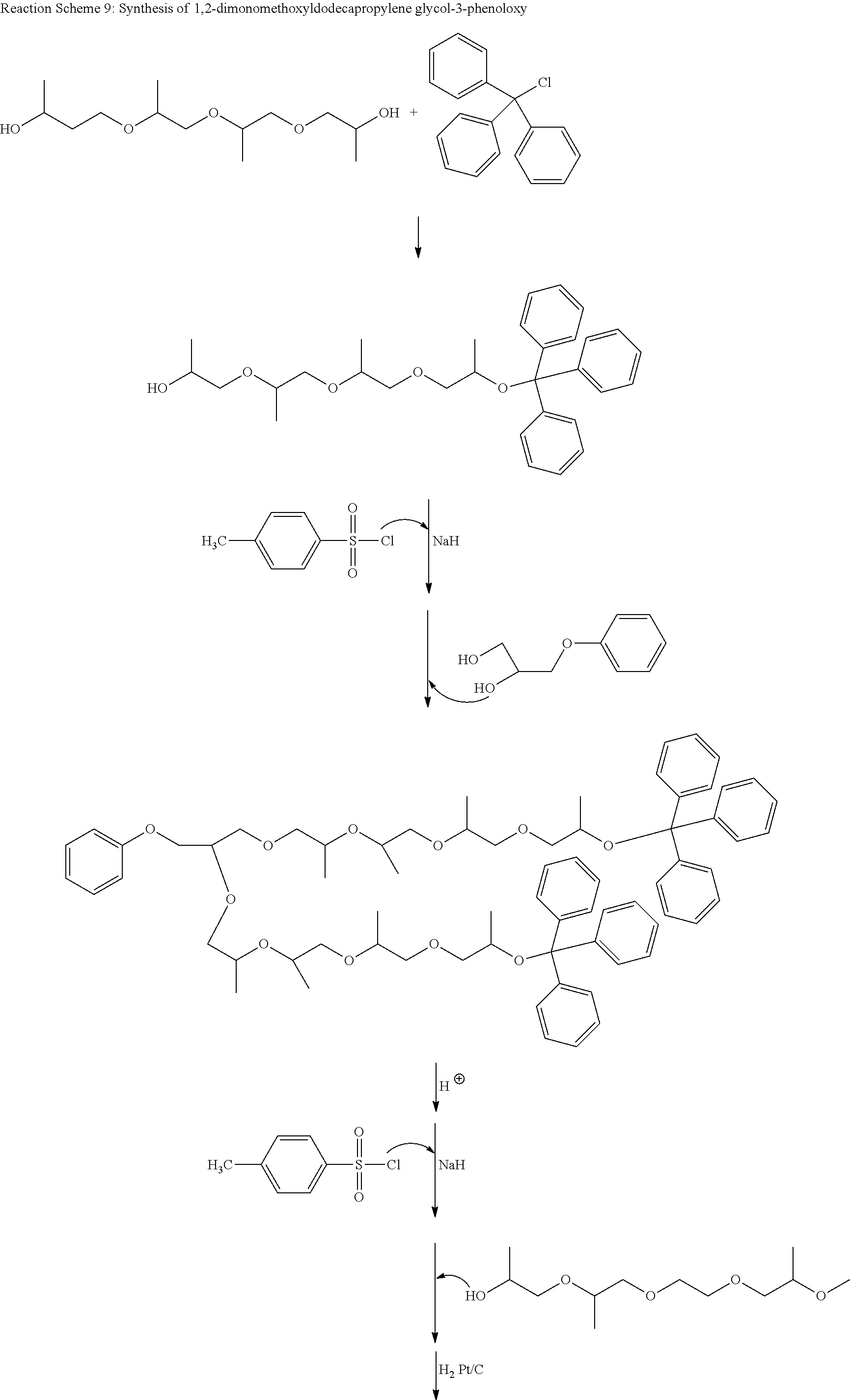

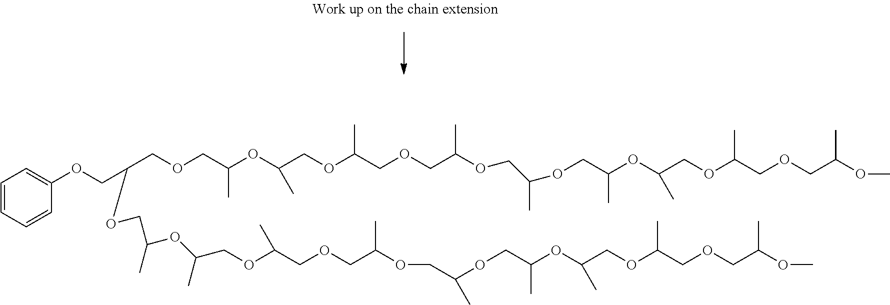

0.15 moles of tetrapropylene glycol was mixed with 0.23 moles of pyridine and heated to 45-50° C. and 0.15 moles of trityl chloride was added. The reaction was carried over night (approximately 16 hours) under constant stifling, then cooled down to room temperature and extracted with toluene. The extract was washed with water, then extracted with hexane and dried over MgSO₄. The solvent was removed under vacuum. A light yellow oily Tr-tetrapropylene glycol was obtained (yield 75 to 85%).

0.1 moles of Tr-tetrapropylene glycol and 0.101 moles of p-toluenesulfonyl chloride were mixed in 100 mL of methylene chloride. The homogeneous mixture was cooled to 0° C. in a dry-ice-acetone bath and 45 g of KOH was added in small portions under vigorous stirring while maintaining the reaction temperature below 5° C. The reaction was completed under constant stirring for 4 hours at 0° C. The crude product was diluted with 100 mL of methylene chloride, then 120 mL of ice-cold water was added. The organic layer was collected, and the aqueous phase was extracted with methylene chloride (2×50 mL). The combined organic layers were washed with water (100 mL) and dried over MgSO₄. The solvent was removed under vacuum to yield (85 to 95%) clear oil.

To a three-necked flask, 1,2-dihydroxyl-rac-glycerol-3-phenoxy (0.1 mol) and NaH (0.4 mol) and dry THF (200 mL) were charged. A dry THF solution (125 mL) of Tr-tetrapropylene glycol tosylate (0.1 mol) was added to the mixture dropwise at room temperature. The mixture was refluxed for 24 hours and then cooled to room temperature. Ice-cold methanol was added to the reaction mixture to quench excessive NaH. The solvent was evaporated and the crude product was extracted with 5% HCl (w/v) and CH₂Cl₂. The crude product was not purified further but taken directly to the next stage of synthesis.

The trityl protecting group was removed by stifling 10 g of 1,2-dihexaethylene glycol-3-phenylglycerol for 3 hours in acidic methanol solution (180 mL MeOH:20 mL, 1 M HCl). The mixture was neutralized with sodium hydrogen carbonate and extracted in chloroform (3×150 mL) and dried over sodium sulfate. Filtration and evaporation of the solvent yields the product (75-80%) of 1,2-di-tetrapropylene glycolglycerol-rac-3-phenoxy.

To a three-necked flask, 1,2-di-tetrapropylene-glycolglycerol-rac-3-phenoxy (0.1 mol) and NaH (0.4 mol) and dry THF (500 mL) were added. A dry THF solution (200 mL) of Tr-tetrapropylene glycol tosylate (0.11 mmol) was added to the mixture dropwise at room temperature. The mixture was refluxed for 24 hours, and cooled to room temperature. Ice-cold methanol was added to the reaction mixture to quench excessive NaH. The solvent was evaporated and the crude product was extracted with 5% HCl (w/v) and CH₂Cl₂.

The above etherification steps were repeated one more time. The solvent was evaporated and further purified by gel permeation chromatography to yield approximately 80% of 1,2 di-trityldodecapropylene glycol-3-benzylglycerol.

The above crude product was transferred to a high pressure resistant glass flask and 200 mL of dry methylene chloride and 10% palladium on carbon (1.5 g). Hydrogenolysis was carried out by purging pure hydrogen at 30° C. in atmosphere for approximately 60 minutes to remove the protective group on the 3′ hydroxyl of the glycerol. After the phenol group was replaced by hydrogen, the solution was cooled to 4 to 6° C. and the catalyst was removed by filtration. Solvent was evaporated to yield 95 to 98% of the intermediate product.

The trityl protecting group was removed by stirring 10 g of 1,2-di-dodecapropylene glycol-3-hydroxylglycerol for 3 hours in acidic methanol solution (180 mL MeOH:20 mL, 1 N HCl). The mixture was neutralized with sodium hydrogen carbonate and extracted in to chloroform (3×150 mL) and dried over sodium sulfate. Filtration and evaporation of the solvent yielded the product (85-90%) of 1,2-di-dodecapropylene glycol-3-hydroxyl-glycerol.

In the above PEG chain extension reaction, the starting PEG reagents preferably comprise 1 to 6 CH₂(CH₃)CH₂O units, and more preferably 3 to 6 CH₂CH₂O units, and more preferably has 4 to 6 CH₂CH₂O units. The reaction between glycerol and the PEG-reagent may occur in the presence or the absence of a linker group. In this embodiment, preferred PEG-reagents have hydroxyl, amino, carboxyl, isocyanate, thiol, carbonate functional groups. Especially preferred PEG-reagents for use in this embodiment of the inventive method include PEG-tosylate, PEG-mesylate and succinyl-PEG. Following the reaction between the glycerol and the PEG-reagent, the protecting groups are removed.

For instance, the starting reagents in the polymer chain extension reaction may be methylene glycol or ethylene glycol or propylene glycol or a mixture of the three from 1 to 6 repeating unit, and more preferably has 3 to 6 repeating unit, and more preferably has 4 to 6 repeating unit. The reaction between glycerol and the reagent may occur in the presence or the absence of a linker group. In this embodiment, preferred polymerization reagents have hydroxyl, amino, carboxyl, thiol, isocyanate, carbonate functional groups. Especially the preferred reagents for use in this embodiment of the inventive method include tosylate, mesylate and succinyl activated intermediates. Following the reaction between the glycerol and the polymerization-reagent, the protecting groups are removed. One of such examples is as showed in Chemical Structure 18.

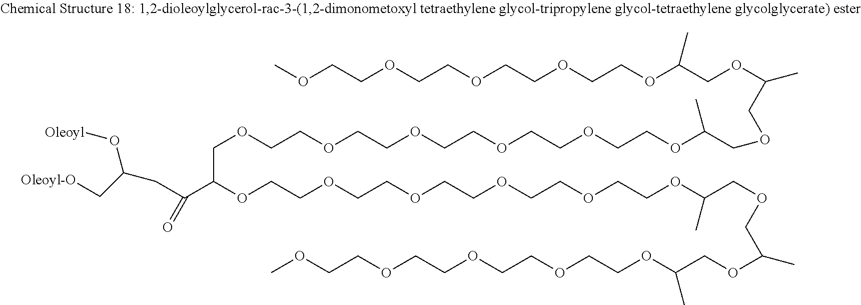

### Example 5

### Solid Dose Compositions

A liquid PEG-lipid conjugate is added to a stainless steel vessel equipped with propeller type mixing blades. The drug substance is added with constant mixing. Mixing continues until the drug is visually dispersed in the lipids at a temperature to 55°-65° C. In a separate container, a PEG-lipid conjugate with a melting temperature above about 30 degrees C. is melted with heating or dissolved in ethanol and added to the vessel with mixing. Mixing continues until fully a homogenous solution is achieved. If necessary, ethanol is removed by vacuum. The solution is filled into capsule shells or predesigned packaging configurations (molds) when the solution is warm. Filled capsules or molds are placed under refrigeration (2-8° C.) until the cream-like mixture is solidified when cooled. A sample formulation is described in Table 5.

TABLE 5

| Ingredient | % |
|---|---|
| Drug Substance | 15 |
| Liquid PEG-lipid Conjugate | 40 |
| Solid PEG-lipid Conjugate | 45 |
| Ethanol | <1 |

The liquid PEG-lipid conjugate may be any branched PEG-lipid where the PEG chains consist of 16 subunits or less. The solid lipid conjugate may be any PEG-lipid where the PEG chains consist of 17 or more subunits.

The drug may be modafinil or nifedapine or esomeprazole or rapamycin or another active agent.

### Example 6

### Solid Dose Compositions

A liquid PEG lipid conjugate (having a melting point below about 15 degrees ° C.) was added to a stainless steel vessel equipped with propeller type mixing blades. The drug substance was added with constant mixing. Mixing continued until the drug was visually dispersed in the lipids at a temperature to 55°-65° C. In a separate container, d-alpha-tocopherol acid polyethylene glycol1000 succinate (TPGS-VE) was dissolved in ethanol and added to the vessel with mixing. Mixing continued until fully a homogenous solution was achieved. Ethanol was be removed by vacuum. The solution was filled into capsule shells or predesigned packaging configuration (molds) when the solution was warm. The filled capsules or molds were placed under refrigeration (2-8° C.). The cream-like mixture was solidified when cooled. A sample formulation is described in Table 6.

TABLE 6

| Ingredient | % |
|---|---|
| Drug Substance (active) | 15 |
| Lipid PEG-lipid Conjugate | 40 |
| TPGS-VE | 45 |
| Ethanol | <1 |

The liquid PEG-lipid conjugate may be any branched PEG-lipid where the PEG chains consisting of 16 subunits or less. The drug may be modafinil or nifedapine or esomeprazole or rapamycin or another active agent.

### Example 7

### Oral Solution Compositions

PEG-lipid was added to a vessel equipped with a mixer propeller. The drug substance was added with constant mixing. Mixing continued until the drug was visually dispersed in the lipids. Pre-dissolved excipients were slowly added to the vessel with adequate mixing. Mixing continued until fully a homogenous solution was achieved. A sample formulation is described in Table 7.

TABLE 7

| Ingredient | mg/mL |
|---|---|
| Drug Substance (active) | 30.0 |
| PEG Lipid | 100 |
| Lactic Acid | 50 |
| Sodium Hydroxide | See below |
| Hydrochloric Acid | See below |
| Sodium Benzoate | 2.0 |
| Artificial Flavor | 5.0 |
| Purified Water | qs 1 mL |

The liquid PEG-lipid conjugate may be any branched PEG-lipid where the PEG chains consisting of 16 subunits or less. Sodium hydroxide is used to prepare a 10% w/w solution in purified water. The targeted pH is in a range of 4.0 to 7.0. NaOH is used to adjust pH if necessary. The drug may be modafinil or nifedapine or esomeprazole or rapamycin or another active agent.

### Example 8

### Cyclosporine Ophthalmic Compositions

PEG-lipid was added to a vessel equipped with a mixer propeller. The cyclosporine drug substance was added with constant mixing. Mixing continued until the drug was visually dispersed in the lipids. Pre-dissolved excipients and sterile purified water were slowly added to the vessel with adequate mixing. Mixing continued until fully a homogenous solution was achieved. A sample formulation is described in Table 8.

TABLE 8

| Ingredient | mg/100 mL |
|---|---|
| Cyclosporine | 50 mg |
| PEG Lipid | 500 |
| Sodium Hydroxide | See below |
| Hydrochloric Acid | See below |
| Sodium Chloride | 900 |
| Sterile purified water | qs 100 mL |

The PEG-lipid conjugate may be any branched PEG-lipid where the PEG chains consisting of 16 subunits or less. Sodium hydroxide is used to prepare a 10% w/w solution in purified water. The targeted pH is in a range of 6.0 to 7.4. NaOH is used to adjust pH if necessary.

### Example 9

### Injection Solution Compositions

The injectable solution was prepared as in Example 7, except that the targeted pH range was between 6.0 and 8.0. A sample formulation is described in Table 9.

TABLE 9

| Ingredient | mg/mL |
|---|---|
| Drug Substance (Active) | 10.0 |
| PEG Lipid | 100 |
| Sodium Hydroxide | See Below |
| Lactic Acid | 20 |
| Purified Water | qs 1 mL |

The lipid may be any branched PEG-lipid where the PEG chains consisting of 16 subunits or less. Sodium hydroxide is used to prepare a 10% w/w solution in purified water. The targeted pH is in a range of 6.5 to 7.4. NaOH is used to adjust pH if necessary. The drug may be triazoles including posaconazole, voriconazole and itraconazole or rapamycin or cyclosporines or tacrolimus or nifedipine or paclitaxel or docetaxel or gefitinib or propofol or rifampin or diazepam or nelfinavir or another active agent.

### Example 10

### Topical Cream Composition

PEG-lipid was added to a stainless steel vessel equipped with propeller type mixing blades. The drug substance was added while mixing. Mixing continued until the drug was visually dispersed in the lipid at a temperature to 60°-65° C. Organic acid, Cholesterol and glycerin were added while mixing. Ethanol and ethyoxydiglycol were added as mixing continued. Finally Carbopol ETD 2020, purified water and triethylamine were added with mixing. Mixing continued until fully a homogenous creamy gel was achieved. The formulation is described in Table 10.

TABLE 10

| Ingredient | % |
|---|---|
| Drug Substance (Active) | 1.0 |
| PEG Lipid | 5.0 |
| Carbopol ETD 2020 | 0.5 |
| Ethyoxydiglycol | 1.0 |
| Ethanol | 5.0 |
| Glycerin | 1.0 |
| Cholesterol | 0.4 |
| Triethylamine | 0.20 |
| Organic acid | 10 |
| Sodium hydroxide | See below |
| Purified water | qs 100 |

The lipid may be any branched PEG-lipid where the PEG chains consisting of 16 subunits or less. Organic acid may be lactic acid or pyruvic acid or glycolic acid. Sodium hydroxide is used to adjust pH if necessary. The targeted pH range was between 3.5 and 7.0. The drug may be itraconazole, posaconazole, voriconazole or equaconazole, Terbinafine, Amorolfine, Naftifine, Butenafine, Benzoic acid, Ciclopirox, Tolnaftate, Undecylenic acid, Flucytosine, Griseofulvin, Haloprogin, Sodium bicarbonate or Fluocinolone acetonide.

### Example 11

### Topical Solution Composition

The topical solution was prepared as in Example 10, a sample formulation is described in Table 11.

TABLE 11

| Ingredient | % |
|---|---|
| Drug Substance (Active) | 1.0 |
| PEG Lipid | 5.0 |
| α-Tocopherol | 0.5 |
| Organic acid | 10.0 |
| Ethanol | 5.0 |
| Sodium Benzoate | 0.2 |
| Sodium Hydroxide | See Below |
| Purified Water | qs 100 |

The lipid may be any branched PEG-lipid where the PEG chains consisting of 16 subunits or less. Organic acid may be lactic acid or pyruvic acid or glycolic acid. Sodium hydroxide is used to adjust pH if necessary. The targeted pH range was between 3.5 and 7.0. The drug may be itraconazole, posaconazole, voriconazole or equaconazole, Terbinafine, Amorolfine, Naftifine, Butenafine, Benzoic acid, Ciclopirox, Tolnaftate, Undecylenic acid, Flucytosine, Griseofulvin, Haloprogin, Sodium bicarbonate or Fluocinolone acetonide.

### Example 12

### Azithromycin Ophthalmic Compositions

PEG-lipid was added to a vessel equipped with a mixer propeller. The azithromycin drug substance was added with constant mixing. Mixing continued until the drug was visually dispersed in the lipids. Pre-dissolved excipients and sterile purified water were slowly added to the vessel with adequate mixing. Mixing continued until fully a homogenous solution was achieved. A sample formulation is described in Table 12.

TABLE 12

| Ingredient | mg/mL |
|---|---|
| Azithromycin | 15 mg |
| PEG Lipid | 150 |
| Sodium Hydroxide | See below |
| Hydrochloric Acid | See below |
| Sodium Chloride | 9 |
| Sterile purified water | qs 1 mL |

The lipid may be any branched PEG-lipid where the PEG chains consisting of 16 subunits or less. Sodium hydroxide is used to prepare a 10% w/w solution in purified water. The targeted pH is in a range of 7.0 to 7.8. NaOH is used to adjust pH if necessary.

Preferable concentration of Azithromycin is 0.5 to 3%, more preferable is 0.5 to 2%, most preferable is 1 to 2%. The preferable ratio of PEG-lipid to the drug (PEG-Lipid/cyclosporine) is 1 to 20, more preferable is 3 to 15, most preferable is 5 to 10.

We claim:

1. A branched polymer-lipid conjugate comprising:
a backbone;
a lipid group covalently attached to the backbone;
a branched polymer having a subchain, said branched polymer being covalently attached to said backbone; and
said polymer subchain having a MW between about 200 and 1200 daltons.

2. The branched polymer-lipid conjugate of claim 1 wherein said polymer subchain is PEG.

3. A chemical composition comprising a plurality of the branched polymer-lipid conjugates of claim 1 with at least about 25 percent oligomer purity.

4. The chemical composition of claim 3 with at least about 50 percent oligomer purity.

5. The branched polymer-lipid conjugate of claim 1 wherein said polymer subchain has a MW greater than about 350 daltons.

6. The branched polymer-lipid conjugate of claim 1 wherein said lipid group is one of a)-f):
a) an alkyl group;
b) a dialkylglycerol ester;
c) selected from the group consisting of Butanoic acid, Hexanoic acid, Octanoic acid, Decanoic acid, Dodecanoic acid, Tetradecanoic acid, Hexadecanoic acid, Octadecanoic acid, Eicosanoic acid, Docosanoic acid, Myristoleic acid, Palmitoleic acid, Oleic acid, Linoleic acid, α-Linolenic acid, Arachidonic acid, and Erucic acid;
d) a bile acid;
e) selected from the group consisting of Cholic acid, Desoxycholic acid, 5-Cholenic acid-3β-ol, Dehydrocholic acid, Glycocholic acid, Glycodeoxycholic acid, Chenodeoxycholic acid, Glycochenodeoxycholic acid, Ursodeoxycholic acid, Lithocholic acid, Hyodeoxycholic acid, and 5β-Cholanic acid-3,7-dione; and
f) cholesterol.

7. The branched polymer-lipid conjugate of claim 1 further comprising a second lipid group covalently attached to said backbone.

8. The branched polymer-lipid conjugate of claim 1 further comprising a linker between said backbone and said polymer.

9. The branched polymer-lipid conjugate of claim 8 wherein said linker is one of a) and b)
a) selected from the group consisting of —S—, —O—, —N—, —OCOO—, carbamoyl-carboxylic acid, n-amino-alkyl-amide, n-hydroxyl-alkyl-amide, alkyl diamide, diamino-carboxylic acid, n-aminoalcohol, diamine, n-amino-alkyl-carbamic acid, n-amino(methyl-thio)ₙ-propanamide, n-mercaptocarboxylic acid, n-mercapto-alpha-aminocarboxylic acid, n-mercapto-alkyl-carbamic acid, Alkyl group, n-mercaptopropylthio)carboxylic acid, Amino-thiol, n-mercapto-alcohol, dithiol, n-amino-(methyl-thio)ₙ-propanoic acid, n-hydroxy-carboxylic acid, n-amino-carboxylic acid, di-carboxylic acid, succinyl, diols, n-hydroxy-alkyl-carbamic acid, and n-hydroxyl-(methyl-thio)ₙ-propanoic acid; and
b) selected from a group consisting of —O—C(O)—, —O—, —S—, and —NH—C(O)—.

10. The branched polymer-lipid conjugate of claim 1 wherein said backbone is one of a)-d):
a) glycerol;
b) selected from the group consisting of 3-amino-1,2-propanediol, 3-bromo-1,2-propanediol, 3-chloro-1,2-propanediol, 3-fluoro-1,2-propanediol, DL-glyceric acid, diaminopropionic acid, tartaric acid, glucoheptonic acid, and 1,2,4-butanetriol, 2,2-Bis(hydroxymethyl)-butyric acid;
c) selected from the group consisting of diethylenetriamine (spermidine), triethylenetriamine (spermine), norspermidine, bis(3-aminopropyl)-1,3-propanediamine, and bis(hexamethylene)triamine; and
d) selected from the group consisting of Lysine, Aspartic Acid, Glutamic Acid, Asparagine, Glutamine, Ornithine, Serine and Threonine.

11. The branched polymer-lipid conjugate of claim 1 comprising two or more said polymers covalently attached to said backbone.

12. The branched polymer-lipid conjugate of claim 1 wherein said polymer subchain is selected from the group consisting of PEG, polymethylene glycol, polypropylene glycol, and copolymers comprised of at least two of the monomers selected from the group consisting of methylene glycol, propylene glycol, and ethylene glycol.

13. A branched polymer-lipid conjugate represented by the formula: 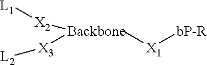
wherein X1, X2 and X3 are same or different linkers, L1 and L2 are the same or different lipids, b is a branched polymer, P is two or more subchains, and R is a terminal group on said branched polymer.

14. The branched polymer-lipid conjugate of claim 13 wherein said lipids have a MW between about 200 and 1200 daltons.

15. A chemical composition comprising a plurality of the branched polymer-lipid conjugates of claim 13 with at least about 25 percent oligomer purity.

16. The branched polymer-lipid conjugate of claim 13 wherein said lipids are one of a)-f):
a) an alkyl group;
b) a dialkylglycerol ester;
c) selected from the group consisting of Butanoic acid, Hexanoic acid, Octanoic acid, Decanoic acid, Dodecanoic acid, Tetradecanoic acid, Hexadecanoic acid, Octadecanoic acid, Eicosanoic acid, Docosanoic acid, Myristoleic acid, Palmitoleic acid, Oleic acid, Linoleic acid, α-Linolenic acid, Arachidonic acid, and Erucic acid;
d) a bile acid;
e) selected from the group consisting of Cholic acid, Desoxycholic acid, 5-Cholenic acid-3β-ol, Dehydrocholic acid, Glycocholic acid, Glycodeoxycholic acid, Chenodeoxycholic acid, Glycochenodeoxycholic acid, Ursodeoxycholic acid, Lithocholic acid, Hyodeoxycholic acid, and 5β-Cholanic acid-3,7-dione; and
f) cholesterol.

17. The branched polymer-lipid conjugate of claim 13 wherein said linkers are one of a) and b):
a) selected from the group consisting of —S—, —O—, —N—, —OCOO—, carbamoyl-carboxylic acid, n-amino-alkyl-amide, n-hydroxyl-alkyl-amide, alkyl diamide, diamino-carboxylic acid, n-aminoalcohol, diamine, n-amino-alkyl-carbamic acid, n-amino(methyl-thio)n-propanamide, n-mercaptocarboxylic acid, n-mercapto-alpha-aminocarboxylic acid, n-mercapto-alkyl-carbamic acid, Alkyl group, n-mercaptopropylthio)carboxylic acid, Amino-thiol, n-mercapto-alcohol, dithiol, n-amino-(methyl-thio)n-propanoic acid, n-hydroxy-carboxylic acid, n-amino-carboxylic acid, di-carboxylic acid, succinyl, diols, n-hydroxy-alkyl-carbamic acid, and n-hydroxyl-(methyl-thio)n-propanoic acid; and
b) selected from a group consisting of —O—C(O)—, —O—, —S—, and —NH—C(O)—.

18. The branched polymer-lipid conjugate of claim 13 wherein said subchains are selected from the group consisting of PEG, polymethylene glycol, polypropylene glycol, and copolymers comprised of at least two of the monomers selected from the group consisting of methylene glycol, propylene glycol, and ethylene glycol.

19. A method for increasing solubility and/or improving pharmacological profile of an active agent with the chemical composition comprising a plurality of the branched polymer-lipid conjugates of claim 13, said method comprising the steps of:
(a) formulating the active agent with the chemical composition comprising a plurality of the branched polymer-lipid conjugates of claim 13, forming a branched polymer-lipid conjugate formulation; and
(b) administering said branched polymer-lipid conjugate formulation to an animal or human.
